(19) United States
(12) Patent Application Publication
(10) Pub. No.: US 2022/0220451 A1
(43) Pub. Date: Jul. 14, 2022

(54) CELL CULTURE DEVICE AND USE OF SAME

(71) Applicant: NATIONAL AGRICULTURE AND FOOD RESEARCH ORGANIZATION, Ibaraki (JP)

(72) Inventor: Toshiaki TAKEZAWA, Ibaraki (JP)

(21) Appl. No.: 17/604,088

(22) PCT Filed: Apr. 15, 2020
(86) PCT No.: PCT/JP2020/016544

§ 371 Date: Oct. 15, 2021

(30) Foreign Application Priority Data
Apr. 18, 2019 (JP) 2019-079522

(51) Int. Cl. C12N 5/071; C12N 5/079; C12N 1/20; C12M 3/00; C12M 1/12; C12M 1/22

(52) U.S. Cl. CPC C12N 5/0697; C12N 5/0671; C12N 5/0621; C12N 1/20; C12N 2513/00; C12M 25/04; C12M 23/44; C12M 23/10; C12N 2533/54; C12M 21/08

(57) ABSTRACT

A cell culture device includes a first culture dish and a second culture dish, the first culture dish has a first membrane on the bottom surface, the second culture dish has a second membrane on the bottom surface, and the first culture dish and the second culture dish are mounted with a gap of which a height is adjustable therebetween.

### TECHNICAL FIELD

The present invention relates to a cell culture device and use of the same. More specifically, the present invention relates to a cell culture device, a tissue type chip, an organ type chip, a cell culture method, a cell transport method, a hepatocyte culture device, a human corneal epithelial model, and a human small intestine model.

Priority is claimed on Japanese Patent Application No. 2019-79522, filed Apr. 18, 2019, the content of which is incorporated herein by reference.

### BACKGROUND ART

Companies related to drug discovery and alternative methods to animal experiment are demanding a “tissue-type culture model” that is closer to a living body than cells in a monolayer culture.

The inventor has developed the basis of a series of products and production technology for annular plastic cell encapsulation devices with both surfaces to which a collagen vitrigel membrane is attached, and proposed ideas to put these into practical use as a non-frozen cell transport tool, a drug discovery support tool, and a cell transplant tool (for example, refer to Patent Literature 1 to 3).

In addition, the inventor has developed an evaluation system for extrapolating biliary excretion of drug metabolites in the human liver by coculturing a cell line of human liver cancer cells in which bile canaliculus-like structures are formed and a cell line of monolayer-cultured human cholangiocarcinoma cells in a collagen vitrigel membrane chamber (for example, refer to Patent Literature 4).

Here, in this specification, when the term “vitrigel” is used, the term “(registered trademark)” may be omitted.

### CITATION LIST

### Patent Literature

[Patent Literature 1]

PCT International Publication No. WO2018/003858
[Patent Literature 2]

PCT International Publication No. WO2018/135252
[Patent Literature 3]

PCT International Publication No. WO2018/211876
[Patent Literature 4]

PCT International Publication No. WO2016/158417

### SUMMARY OF INVENTION

### Technical Problem

Based on the above development results, the inventor has aimed to develop a better cell culture device.

The present invention has been made in view of the above circumstances and provides a cell culture device having excellent handling.

### Solution to Problem

The present invention includes the following aspects.
[1] A cell culture device including a first culture dish and a second culture dish,

wherein the first culture dish has a first membrane on the bottom surface,

wherein the second culture dish has a second membrane on the bottom surface, and

wherein the first culture dish and the second culture dish are mounted with a gap of which a height is adjustable therebetween.
[2] The cell culture device according to [1],

wherein the first culture dish and the second culture dish are mounted by engagement or fitting.
[3] The cell culture device according to [1] or [2],

wherein the bottom surface of the first culture dish has a tapered structure and a marking line for preventing the first membrane from bending, and/or the bottom surface of the second culture dish has a tapered structure and a marking line for preventing the second membrane from bending.
[4] The cell culture device according to any one of [1] to [3],

wherein the first culture dish has a female screw structure,

wherein the second culture dish has a male screw structure, and

wherein the first culture dish and the second culture dish are mounted by screwing.
[5] The cell culture device according to any one of [1] to [4],

wherein the first culture dish has a hole at the height of the gap on a side surface.
[6] The cell culture device according to any one of [1] to [5],

wherein at least one of the first membrane and the second membrane is a liquid-permeable porous membrane or a semipermeable membrane having liquid-tightness in a gas phase and semipermeability in a liquid phase.
[7] The cell culture device according to [6],

wherein the semipermeable membrane contains a gelling extracellular matrix component.
[8] The cell culture device according to [7],

wherein the gelling extracellular matrix component is collagen.
[9] The cell culture device according to any one of [1] to [8], further including

an Nth culture dish (N is an integer of 3 or more),

wherein the Nth culture dish has an Nth membrane on the bottom surface, and

wherein the (N−1)th culture dish and the Nth culture dish are mounted with a gap of which a height is adjustable therebetween.
[10] The cell culture device according to any one of [1] to [9],

wherein a buffer part is provided on the bottom surface of the first culture dish.
[11] The cell culture device according to any one of [1] to [10],

wherein a lid for closing the second culture dish is provided.
[12] A tissue type chip including the cell culture device according to any one of [1] to [11] in which at least the first culture dish contains one type of cells.
[13] An organ type chip including the cell culture device according to any one of [1] to [11] including a culture dish containing different types of cells.
[14] A cell culture method using the cell culture device according to any one of [1] to [11].
[15]A cell transport method using the cell culture device according to any one of [1] to [9].
[16] A hepatocyte culture device that promotes accumulation and excretion of hepatic metabolites in bile canaliculus-like structures, the device including:

a first cell culture product containing a plurality of hepatocytes;

a second culture dish which is able to construct bile canaliculus-like structures between hepatocytes in the first cell culture product, and has a membrane that a physiologically active substance is able to permeate on the bottom surface and in which the first cell culture product is accommodated;

a second cell culture product that is able to increase an excretion activity of hepatic metabolites in the first cell culture product; and

a first culture dish in which the second cell culture product is accommodated,

wherein the first cell culture product is placed on the second cell culture product and co-cultured, and

wherein the first culture dish and the second culture dish are mounted with a gap of which a height is adjustable therebetween.
[17] The hepatocyte culture device according to [16],

wherein the excretion activity of the hepatic metabolites is an activity of excreting hepatic metabolites accumulated in the bile canaliculus-like structures between hepatocytes and in the hepatocytes in the first cell culture product.
[18] The hepatocyte culture device according to [16] or [17],

wherein the hepatocytes are cultured hepatocytes derived from nonnal liver tissues, liver cancer tissues or stem cells selected from the group consisting of humans, rats, monkeys, apes, cats, dogs, pigs, cows, sheep, horses, chickens and ducks.
[19] The hepatocyte culture device according to any one of [16] to [18],

wherein the hepatocytes are HepG2-NIAS cells (RCB4679 strain).
[20] The hepatocyte culture device according to any one of [16] to [19],

wherein the second cell culture product is a culture product of cells derived from epithelium, mesenchyme or endothelium selected from the group consisting of humans, rats, monkeys, apes, cats, dogs, pigs, cows, sheep, horses, chickens and ducks.
[21] The hepatocyte culture device according to any one of [16] to [20],

wherein a membrane that is permeable to the physiologically active substance contains a gelling extracellular matrix component.
[22] The hepatocyte culture device according to [21],

wherein the gelling extracellular matrix component is collagen.
[23] A human corneal epithelial model, including:

a first cell culture product containing a plurality of human corneal epithelial cells;

a first culture dish which has a membrane that a physiologically active substance in the first cell culture product is able to permeate on the bottom surface and in which the first cell culture product is accommodated; and

a second culture dish having a filtration membrane on the bottom surface,

wherein the second culture dish is placed on the first culture dish, and

wherein the first culture dish and the second culture dish are mounted with a gap of which a height is adjustable therebetween.
[24] The human corneal epithelial model according to [23],

wherein a buffer part is provided on the bottom surface of the first culture dish.
[25] The human corneal epithelial model according to [23] or [24],

wherein a lid for closing the second culture dish is provided.
[26] A human small intestine model, including

an anaerobic bacterial culture product;

a second culture dish having a sterile filtration membrane on the bottom surface in which the anaerobic bacterial culture product is accommodated;

a small intestine-derived cell culture product;

a first culture dish which has a semipermeable membrane having liquid-tightness in a gas phase and semipermeability in a liquid phase on the bottom surface and in which the small intestine-derived cell culture product is accommodated; and

an oxygen supply mechanism,

wherein the anaerobic bacterial culture product is placed on the small intestine-derived cell culture product, the small intestine-derived cell culture product is placed on the oxygen supply mechanism, and the first culture dish and the second culture dish are mounted with a gap of which a height is adjustable therebetween.
[27] The human small intestine model according to [26],

wherein the oxygen supply mechanism includes a mechanism that uses oxygen in air or an oxygen generation mechanism.

### Advantageous Effects of Invention

According to the present invention, it is possible to provide a cell culture device having excellent handling.

### BRIEF DESCRIPTION OF DRAWINGS

FIG. 1(a) is a perspective view of a cell culture device of the present embodiment. FIG. 1(b) is a side view of the cell culture device of the present embodiment. FIG. 1(c) is a side view of the cell culture device of the present embodiment. FIG. 1(d) is a side view of a first culture dish. FIG. 1(e) is a top view of the first culture dish. FIG. 1(f) is a side view of a second culture dish. FIG. 1(g) is a top view of the second culture dish.

FIG. 2(a) is a side view of a protection part that covers a first membrane. FIG. 2(b) is a perspective view of a lid. FIG. 2(c) is a side view of the cell culture device of the present embodiment. FIG. 2(d) is a side view of the cell culture device of the present embodiment.

FIG. 3 is a perspective view of a pedestal.

FIGS. 4(a) to 4(c) are side views of a hepatocyte culture device of the present embodiment.

FIG. 5(a) is a bottom view of the first culture dish. FIG. 5(b) is a side view of leg parts.

FIG. 6(a) is a side view of the second culture dish. FIG. 6(b) is a side view of the first culture dish. FIG. 6(c) is a bottom view of the second culture dish. FIG. 6(d) is a bottom view of the first culture dish.

FIG. 7 is a side view of an oxygen partial pressure control model of the present embodiment.

FIG. 8 is a side view of a human small intestine model of the present embodiment.

FIG. 9(a) shows images showing a process of producing vinyl cut to φ11 mm and φ14 mm. FIG. 9(b) shows images of acrylic jigs for a male screw and a female screw.

FIG. 10(a) shows images showing a process of bonding vinyl to an acrylic jig A and combining acrylic jigs A and B. FIG. 10(b) shows images showing a process of injecting collagen sol into a male screw jig.

FIG. 11(a) shows images showing a process of injecting collagen sol into a female screw jig. FIG. 11(b) shows images showing a gelation process of the injected collagen sol.

FIG. 12(a) shows images showing a vitrification process of the gelled collagen. FIG. 12(b) shows images showing a process of rehydration of a vitrified collagen gel membrane.

FIG. 13(a) shows images showing a collagen vitrigel membrane after rehydration. FIG. 13(b) shows images showing a re-vitrification process of the rehydrated collagen vitrigel membrane.

FIG. 14(a) shows images showing a process of releasing a jig AB of a male screw jig. FIG. 14(b) shows images showing a process of releasing a jig AB of a female screw jig.

FIG. 15(a) shows images showing a process of bonding a collagen vitrigel membrane to a screw type member. FIG. 15(b) shows images showing a process of trimming a collagen vitrigel membrane protruding to the outside of the screw type member.

FIG. 16(a) shows images of legs for a screw type member and a jig. FIG. 16(b) shows images showing a process of bonding legs for a screw type member.

FIG. 17(a) shows images showing a process of combining a male screw and a female screw. FIG. 17(b) shows images showing a silicone O-ring-mounting process.

FIG. 18(a) is an image showing a tip surface of a male screw with a marking line and an angle of 2 degrees before a collagen vitrigel membrane is attached. FIG. 18(b) is an image showing a tip surface of a female screw with a marking line and an angle of 2 degrees before a collagen vitrigel membrane is attached. FIG. 18(c) is an image of a male screw and a female screw with a tip surface with a marking line and an angle of 2 degrees to which a collagen vitrigel membrane is attached.

FIG. 19(a) is an image of a male screw with a silicone O-ring mounted thereon. FIG. 19(b) is an image of a female screw with legs bonded thereto. FIG. 19(c) is an oblique image of a cell culture device in which a male screw with a silicone O-ring mounted thereon and a female screw with legs bonded thereto are screwed. FIG. 19(d) is a side image of the cell culture device in which a male screw with a silicone O-ring mounted thereon and a female screw with legs bonded thereto are screwed.

FIG. 20(a) shows a male screw with no tilt angle (0 degrees). FIG. 20(b) shows a male screw with a tilt angle of 2 degrees. FIG. 20(c) shows a female screw with no tilt angle (0 degrees). FIG. 20(d) shows a female screw with a tilt angle of 2 degrees.

### DESCRIPTION OF EMBODIMENTS

Hereinafter, embodiments of the present invention will be described in detail with reference to the drawings in some cases. Here, in the drawings, the same or corresponding components will be denoted with the same or corresponding reference numerals, and redundant descriptions thereof will be omitted. Here, the dimensional ratios in the drawings may be exaggerated for explanation, and do not necessarily match actual dimensional ratios.

### [Cell Culture Device]

In one embodiment, the present invention provides a cell culture device including a first culture dish and a second culture dish. The first culture dish has a first membrane on the bottom surface. The second culture dish has a second membrane on the bottom surface. The first culture dish and the second culture dish are mounted such that the height of the gap is adjustable.

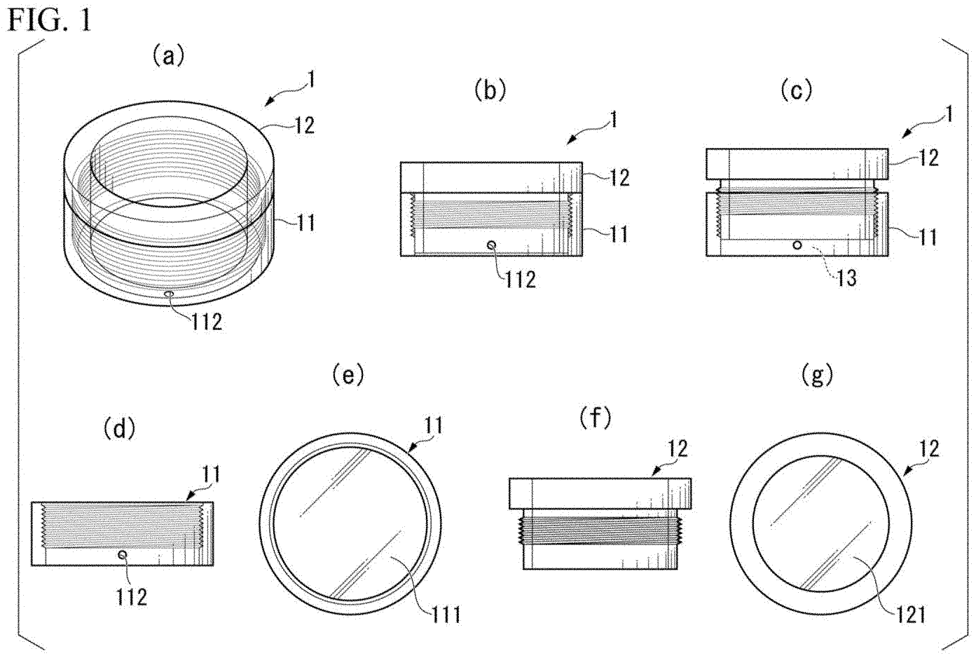

FIGS. 1(a) to 1(b) are schematic views illustrating a structure of an example of the cell culture device of the present embodiment. FIG. 1(a) is a perspective view of the cell culture device of the present embodiment. FIG. 1(b) is a side view of FIG. 1(a).

As shown in FIGS. 1(a) to 1(b), in a cell culture device 1, a first culture dish 11 and a second culture dish 12 are mounted such that the height of the gap is adjustable. FIG. 1(c) is a side view when the state of the second culture dish 12 at the lowermost position changes to the state of the second culture dish 12 pulled up from the first culture dish 11 in the cell culture device 1. As shown in FIG. 1(c), when the second culture dish 12 is pulled up from the first culture dish 11, a gap 13 is formed. The height of the gap 13 can be adjusted by adjusting the distance between the first culture dish 11 and the second culture dish 12, and is, for example, preferably 0 cm to 1 cm, and more preferably 0 cm to 0.5 cm. According to the application of the cell culture device 1, for example, the height of the gap 13 can be adjusted according to the volume of cells to be encapsulated.

FIG. 1(d) is a side view of the first culture dish 11. In FIG. 1(d), the first culture dish 11 has a female screw structure in which a female screw is cut on an inner circumferential surface. FIG. 1(e) is a top view of the first culture dish 11. In FIG. 1(e), the first culture dish 11 has a first membrane 111 on the bottom surface.

FIG. 1(f) is a side view of the second culture dish 12. In FIG. 1(f), the second culture dish 12 has a male screw structure in which a male screw is cut on an outer circumferential surface. FIG. 1(g) is a top view of the second culture dish 12. In FIG. 1(g), the second culture dish 12 has a second membrane 121 on the bottom surface.

As a method of adjusting the height of the gap formed between the first culture dish 11 and the second culture dish 12, a silicone O-ring, a metal washer, a cyclic nylon film or the like may be mounted on the base of the male screw structure and then screwed to the female screw.

The first culture dish 11 and the second culture dish 12 may be mounted with a gap of which a height is adjustable therebetween, and are preferably mounted by engagement or fitting.

In FIGS. 1(a) to 1(b), the first culture dish 11 and the second culture dish 12 are mounted by screwing, but they may be mounted via a tapered structure regardless of a mounting mechanism.

The outer diameters of the first culture dish 11 and the second culture dish 12 are preferably substantially the same. The outer diameter is preferably 6 mm to 100 mm, more preferably 10 mm to 60 mm, and still more preferably 14 mm to 30 mm. The inner diameter of the first culture dish 11 is preferably 2 mm to 96 mm, more preferably 6 mm to 56 mm, and still more preferably 10 mm to 26 mm. The inner diameter of the second culture dish 12 is preferably 1 mm to 80 mm, more preferably 2 mm to 40 mm, and still more preferably 3 mm to 25 mm.

In addition, the height of the cell culture device 1 is preferably 0.5 cm to 10 cm, and more preferably 1 cm to 5 cm.

The internal volume of the cell culture device 1 of the present embodiment may be small enough to inject cells suspended in a culture medium and construct a multicellular structure used in an in vitro test system. Specifically, for example, the internal volume is preferably 10 mL or less, more preferably 10 μL to 5 mL, still more preferably 15 μL to 2 mL, and particularly preferably 20 μL to 1 mL. When the internal volume is the upper limit value or less, oxygen, and nutrients of the culture medium are sufficiently supplied, and the cells can be efficiently cultured for a long time. In addition, when the internal volume is the lower limit value or more, cells with sufficient cell number and cell density used in the in vitro test system can be obtained.

In this specification, “multicellular structure” is monolayer cells in which a plurality of cells formed cell-substratum bonds and cell-cell bonds or a 3D structure composed of multilayer cells. The multicellular structure in the present embodiment is composed of one or more types of functional cells and a substratum that acts as a scaffold therefor. That is, the multicellular structure in the present embodiment constructs a morphology more similar to a tissue or organ in a living body due to an interaction of a plurality of functional cells and a substratum. Therefore, in the multicellular structure, capillary network-like structures such as blood vessels and/or bile ducts may be three-dimensionally constructed. Such a capillary network-like structure may be formed only inside the multicellular structure, and at least a part thereof may be formed so that it is exposed to the surface or the base of the multicellular structure.

On the side surface of the first culture dish 11, it is preferable to have a hole 112 at a height of the gap required for encapsulating cells (refer to FIG. 1(b)). After a cell suspension is added to the first culture dish 11, when the second culture dish 12 is mounted, an excess culture medium can be released from the hole 112.

Examples of membranes used for the first culture dish 11 and the second culture dish 12 include a porous membrane.

In this specification, “porous membrane” is a membrane having a plurality of pores, and includes a membrane having voids and a membrane having pores and voids.

A liquid-permeable porous membrane used in the cell culture device of the present embodiment may be a membrane having pores such that cells encapsulated inside do not permeate to the outside, but there is no particular limitation thereto. Examples of porous membranes include filter paper, a semipermeable membrane (for example, an ultrafiltration membrane, etc.), a non-woven fabric, a gauze-like mesh, and various membrane filters, but the present invention is not limited thereto.

In addition, the size of the pores of the porous membrane in the present embodiment is, for example, preferably 0.01 μm to 1,500 μm, for example, preferably 0.01 μm to 1.0 μm, and for example, preferably 0.01 μm to 0.45 μm. The size of the pores may be appropriately selected according to the size of cells to be encapsulated therein.

Among these, the porous membrane in the present embodiment is preferably a semipermeable membrane having liquid-tightness in a gas phase and semipermeability in a liquid phase. Since the semipermeable membrane has liquid-tightness in a gas phase, for example, when the cell culture device of the present embodiment contains a liquid such as a culture medium, in the gas phase, the liquid does not leak and can be kept inside. The liquid-tightness is due to surface tension on the semipermeable membrane. On the other hand, when the device contains a liquid, since a gas can pass therethrough, the inside liquid evaporates over time.

The semipermeable membrane used in the present embodiment may be, for example, a membrane that allows permeation of a polymer compound having a molecular weight of about 1,000,000 or less, and may be, for example, a membrane that allows permeation of a molecular compound having a molecular weight of about 200,000 or less.

In this specification, “liquid-tightness” means a state in which a liquid does not leak.

In addition, in this specification, “semipermeability” means a property in which only molecules or ions having a certain molecular weight or less are able to permeate, and a “semipermeable membrane” is a membrane having such a property.

The material of the porous membrane is preferably a material having no cytotoxicity, and may be a natural polymer compound or a synthetic polymer compound. In addition, when the porous membrane is a semipermeable membrane, the material thereof is preferably a material having biocompatibility.

Here, in this specification, “biocompatibility” is an evaluation criterion indicating compatibility between living tissues and materials. In addition, “having biocompatibility” means a state in which the material does not have toxicity, does not have microorganism-derived components such as endotoxins, does not physically stimulate the living tissue, and is not rejected even if it interacts with proteins, cells and the like constituting the living tissue.

Examples of natural polymer compounds include a gelling extracellular matrix-derived component, polysaccharides (for example, arginate, cellulose, dextran, pullulane, polyhyaluronic acid, derivatives thereof, etc.), chitin, poly(3-hydroxy alkanoate) (particularly, poly(β-hydroxybutyrate), poly(3-hydroxyoctanoate)), poly(3-hydroxy fatty acid), fibrin, agar, and agarose, but the present invention is not limited thereto.

Cellulose includes those modified by synthesis, and examples thereof include cellulose derivatives (for example, alkyl cellulose, hydroxyalkyl cellulose, cellulose ether, cellulose ester, nitrocellulose, chitosan, etc.). Examples of more specific cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salts.

Among these, as the natural polymer compound, a gelling extracellular matrix-derived component, fibrin, agar, or agarose is preferable because it has excellent water retention.

Examples of gelling extracellular matrix-derived components include collagen (type I, type II, type III, type V, type XI, etc.), basement membrane components reconstituted from mouse EHS tumor extract (including type IV collagen, laminin, heparan sulfate proteoglycan, etc.) (product name: Matrigel), glycosaminoglycan, hyaluronic acid, proteoglycan, and gelatin, but the present invention is not limited thereto. It is possible to produce a porous membrane (particularly, a semipermeable membrane) by selecting components such as optimal salts for respective gelation, the concentration thereof, the pH, and the like. In addition, when raw materials are combined, it is possible to obtain a porous membrane (particularly, a semipermeable membrane) that imitates various in vivo tissues.

When the material of the porous membrane in the present embodiment is an extracellular matrix-derived component, it is preferable that it contain 0.1 mg to 10.0 mg and more preferable that it contain 0.5 mg to 5.0 mg of the extracellular matrix-derived component per unit area 1 cm² of the porous membrane (particularly, a semipermeable membrane). In particular, when the extracellular matrix-derived component is atelocollagen, it is preferable that it contain 0.5 mg to 10.0 mg, and more preferable that it contain 2.5 mg to 5.0 mg of atelocollagen per unit area 1 cm² of the porous membrane (particularly, a semipermeable membrane).

When the content of the extracellular matrix-derived component (particularly, atelocollagen) in the porous membrane (particularly, a semipermeable membrane) is within the above range, a more preferable strength for culturing cells can be obtained.

Here, “weight per unit area 1 cm² of the membrane” refers to a weight of the component contained in 1 cm² of the material piece with an arbitrary thickness of the membrane.

Examples of synthetic polymer compounds include polyphosphazene, poly(vinyl alcohol), polyamide (for example, nylon, etc.), polyester amide, poly(amino acid), polyanhydride, polysulfone, polycarbonate, polyacrylate (acrylic resin), polyalkylene (for example, polyethylene, etc.), polyacrylamide, polyalkylene glycol (for example, polyethylene glycol, etc.), polyalkylene oxide (for example, polyethylene oxide, etc.), polyalkylene terephthalate (for example, polyethylene terephthalate, etc.), polyorthoester, polyvinyl ether, polyvinyl ester, polyvinyl halide, polyvinylpyrrolidone, polyester, polysiloxane, polyurethane, polyhydroxyic acid (for example, polylactide, polyglycolide, etc.), poly(hydroxybutyric acid), poly(hydroxyvaleric acid), poly[lactide-co-(ε-caprolactone)], poly[glycolide-co-(ε-caprolactone)], etc.), and poly(hydroxy alkanoate), and copolymers thereof, but the present invention is not limited thereto.

More specific examples of polyacrylate (acrylic resin) include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Among these, regarding the synthetic polymer compound, polyhydroxy acid (for example, polylactide, polyglycolide, etc.), polyethylene terephthalate, poly(hydroxybutyric acid), poly(hydroxyvaleric acid), poly[lactide-co-(ε-caprolactone)], poly[glycolide-co-(ε-caprolactone)], etc.), poly(hydroxy alkanoate), polyorthoester, or a copolymer is preferable.

The material of the porous membrane in the present embodiment may be composed of one or more types of the materials exemplified above. In addition, the material of the porous membrane in the present embodiment may be composed of either a natural polymer compound or a synthetic polymer compound, or may be composed of both a natural polymer compound and a synthetic polymer compound.

Among these, when the porous membrane in the present embodiment is a semipermeable membrane, the material thereof is preferably a natural polymer compound, more preferably a gelling extracellular matrix-derived component, and still more preferably collagen. In addition, regarding a more preferable raw material among collagen, native collagen or atelocollagen may be exemplified.

In addition, examples of materials constituting the porous membrane include a hydrogel. In this specification, “hydrogel” refers to a substance in which a polymer compound has a mesh structure due to chemical bonds, and the mesh retains a large amount of water. More specifically, the hydrogel is a substance obtained by introducing crosslinks to an artificial material of a natural product polymer compound or a synthetic polymer compound and gelling it.

Examples of hydrogels include natural polymer compounds such as the above gelling extracellular matrix-derived components, fibrin, agar, agarose, and cellulose, and synthetic polymer compounds such as polyacrylamide, polyvinyl alcohol, polyethylene oxide, and poly(II-hydroxyethylmethacrylate)/polycaprolactone.

More specifically, as a method of producing a porous membrane using a hydrogel, first, a hydrogel that is not completely gelled (hereinafter referred to as “sol”) is placed in a mold, and gelation is induced.

When the sol is a collagen sol, a collagen sol prepared using saline, phosphate buffered saline (PBS), Hank's balanced salt solution (HBSS), a basal culture medium, a serum-free culture medium, a serum-containing culture medium or the like with an optimal salt concentration may be used. In addition, the pH of the collagen sol during gelation may be, for example, 6 or more and 8 or less.

In particular, when a serum-free culture medium is used, since it is possible to prevent substances (for example, an antigen, a pathogenesis factor, etc.) that are contained in other animal serum components and are not suitable for transplantation from being incorporated into the porous membrane, it is possible to obtain a multicellular structure suitable when cells cultured in a cell culture device are used for transplantation.

In addition, the collagen sol may be prepared, for example, at about 4° C. Then, for heat retention during gelation, the temperature may be lower than the denaturation temperature of collagen depending on the animal species of collagen used, and generally, the temperature is kept at 20° C. or higher and 37° C. or lower, and thus gelation can be performed for several minutes to several hours.

In addition, the concentration of the collagen sol for producing a porous membrane is preferably 0.1% to 1.0%, and more preferably 0.2% to 0.6%. When the concentration of the collagen sol is the lower limit value or more, gelation is not too weak, and when the concentration of the collagen sol is the upper limit value or less, a porous membrane (particularly, a semipermeable membrane) made of a uniform collagen gel can be obtained.

In addition, the obtained hydrogel may be dried to obtain a dried hydrogel product. When the hydrogel is dried, free water in the hydrogel can be completely removed, and additionally, bound water can be partially removed.

In addition, the obtained dried hydrogel product may be rehydrated with PBS, a culture medium to be used, or the like to obtain vitrigel (registered trademark).

As the time period of the vitrification process (the process of proceeding with the partial removal of bound water after free water in the hydrogel is completely removed) is longer, vitrigel (registered trademark) having excellent transparency and strength can be obtained when rehydrated. Here, as necessary, vitrigel (registered trademark) obtained by rehydration after vitrification for a short period can be washed with PBS or the like, and vitrified again.

As a drying method, various methods, for example, air drying, drying in a closed container (circulating air in the container, and constantly supplying dry air), drying in an environment in which silica gel is provided, and the like can be used. For example, as an air drying method, a method of performing drying in an incubator kept aseptic at 10° C. 40% humidity for 2 days or performing drying on an aseptic clean bench at room temperature over a day and night may be exemplified.

Here, in this specification, “vitrigel (registered trademark)” refers to a gel in a stable state obtained by vitrifying a conventional hydrogel and then rehydrating it, and has been named “vitrigel (registered trademark)” by the inventors.

In addition, in this specification, in explaining in detail a process of producing a porous membrane made of a hydrogel, a dried product of a hydrogel immediately after the vitrification process that has not undergone the rehydration process is simply referred to as a “dried hydrogel product.” Then, the gel obtained through the rehydration process after the vitrification process is distinguished and represented as “vitrigel (registered trademark).” In addition, the dried product obtained by vitrifying the vitrigel (registered trademark) is referred to as a “vitrigel (registered trademark) dried product.” In addition, a product obtained by subjecting the vitrigel (registered trademark) dried product to a process of irradiating UV rays is referred to as a “vitrigel (registered trademark) dried product subjected to a UV irradiation treatment.” In addition, a gel obtained by subjecting the “vitrigel (registered trademark) dried product subjected to a UV irradiation treatment” to a process of rehydrating is referred to as a “vitrigel (registered trademark) material.” In addition, a dried product obtained by vitrifying the vitrigel (registered trademark) material is referred to as a “dried product of vitrigel (registered trademark) material.” Therefore, “vitrigel (registered trademark)” and “vitrigel (registered trademark) material” are hydrates.

In order to irradiate UV rays, a known UV irradiation device can be used.

Regarding the irradiation energy of UV rays to the vitrigel (registered trademark) dried product, a total irradiation amount per unit area is preferably 0.1 mJ/cm² to 6,000 mJ/cm² or less, more preferably 10 mJ/cm² to 4,000 mJ/cm², and still more preferably 100 mJ/cm² to 3,000 mJ/cm². When the total irradiation amount is within the above range, the transparency and strength of the vitrigel (registered trademark) material obtained in the subsequent rehydration process can be made particularly suitable.

The thickness of the porous membrane in the present embodiment is not particularly limited, and is preferably 1 μm to 1,000 μm, more preferably 1 μm to 500 μm, still more preferably 5 μm to 300 μm, and particularly preferably 10 μm to 200 μm. When the thickness of the porous membrane is within the above range, it is possible to obtain a more preferable strength for culturing cells.

Examples of materials of the cell culture device other than the porous membrane include glass materials such as soda-lime glass, Pyrex (registered trademark) glass, Vycor (registered trademark) glass, and quartz glass; elastomer materials such as urethane rubber, nitrile rubber, silicone rubber, silicone resin (for example, polydimethylsiloxane), fluororubber, acrylic rubber, isoprene rubber, ethylene propylene rubber, chlorosulfonated polyethylene rubber, epichlorohydrin rubber, chloroprene rubber, styrene butadiene rubber, butadiene rubber, and polyisobutylene rubber; plastics containing polymers such as poly(vinyl chloride), poly(vinyl alcohol), poly(methyl methacrylate), poly(vinyl acetate-co-maleic anhydride), poly(dimethylsiloxane) monomethacrylate, cyclic olefin polymer, fluorocarbon polymer, polystyrene, polypropylene, polyethylenimine, and polyethylene terephthalate (PET); and copolymers such as poly(vinyl acetate-co-maleic anhydride), poly(styrene-co-maleic anhydride), poly(ethylene-co-maleic acid), or derivatives thereof, and a plastic is preferable. In addition, the plastic may be coated with silicone.

The color of the cell culture device is not particularly limited, but it is preferable to appropriately select a transparent color and an opaque (light-shielding) color using various microscopes in order to observe cells. In addition, the cell culture device may be devised (for example, coloring, printing, etc.) in order to identify individual cells to be cultured.

Examples of a method of producing a cell culture device other than the porous membrane include a compression molding method, an injection molding method, and an extrusion molding method, but the present invention is not limited thereto.

In the cell culture device of the present embodiment, at least one of the first membrane and the second membrane is preferably a semipermeable membrane having liquid-tightness in a gas phase and having semipermeability in a liquid phase. Examples of combinations of membranes in the cell culture device include a combination in which a first membrane is a vitrigel membrane and a second membrane is a plastic film such as PET, a combination in which a first membrane is a plastic film such as PET and a second membrane is a vitrigel membrane, a combination in which both a first membrane and a second membrane are a vitrigel membrane, a combination in which a first membrane is a sterile filtration membrane and a second membrane is a vitrigel membrane, a combination in which a first membrane is a plastic film such as PET and a second membrane is a sterile filtration membrane, and a combination in which a first membrane is a vitrigel membrane and a second membrane is a dialysis membrane. Examples of sterile filtration membranes include those having a pore size of 0.22 μm and 0.45 μm.

In addition, a combination in which both the first membrane and the second membrane are a plastic film such as PET may be exemplified.

When the material of the cell culture device other than the membrane and the material of the membrane are the same, they may be regarded as being integrated. For example, when the material of the cell culture device other than the membrane and the material of the membrane are a plastic resin such as PET, they are included in the scope of the present invention without separately applying a plastic film such as PET.

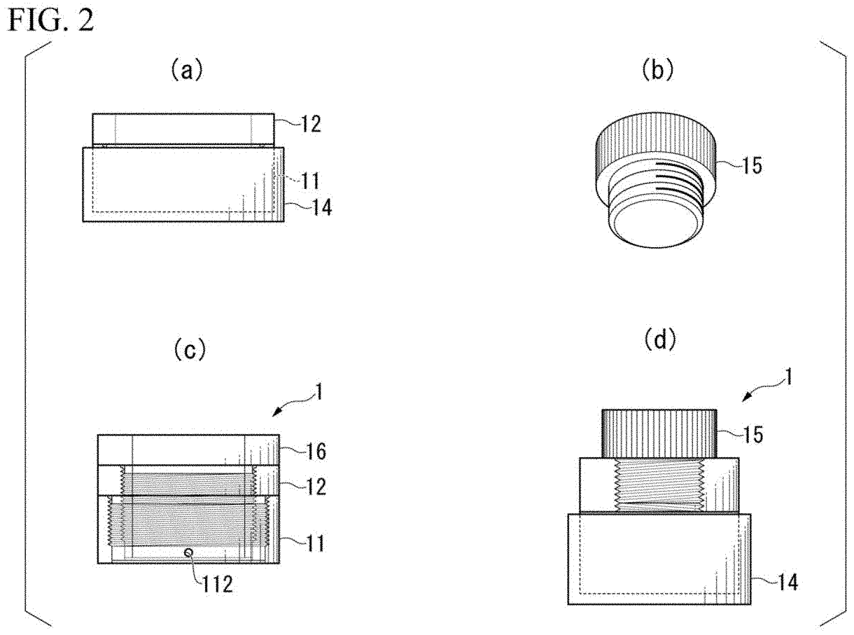

When a semipermeable membrane such as a vitrigel membrane is used for the first membrane 111, the cell culture device 1 may have a protection part 14 that covers the first membrane 111 (refer to FIG. 2(a)). Examples of materials of the protection part 14 are the same as those of the materials of the cell culture device other than the membrane described above. The protection part 14 preferably has a female screw structure for adjusting a gap between it and the first membrane 111. This gap may be filled with a culture medium.

In addition, when a semipermeable membrane such as a vitrigel membrane is used for the first membrane 111, the membrane without the protection part 14 may be placed in a multi-well plate including a culture medium. In this case, in order to promote contact between the first membrane 111 and the culture medium in the multi-well plate, a buffer part such as a mesh may be provided on the outer surface of the membrane 111.

In addition, when cells are cultured in the second culture dish, a lid 15 that closes the second culture dish may be provided (refer to FIG. 2(b)). The lid 15 preferably has a male screw structure. The male screw structure is preferably a structure in which excess gas or liquid is discharged from the gap between the screw and the second culture dish in order to release the pressure while the screw is tightened. In addition, the second culture dish may have a hole on the side surface.

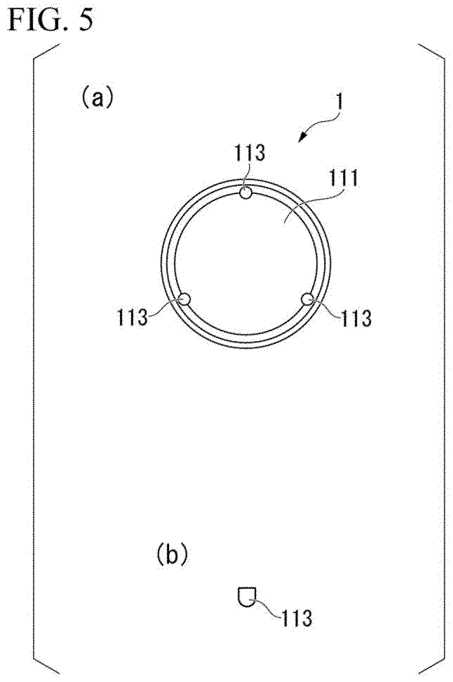

In addition, when a semipermeable membrane such as a vitrigel membrane is used for the first membrane 111, and the cell culture device 1 is placed in the multi-well plate including a culture medium, the first culture dish may have a plurality of leg parts 113 on the bottom surface. FIG. 5(a) is a top view of the cell culture device 1 when viewed from the top side. The number of leg parts 113 is preferably 3 or more and more preferably 3. When a culture dish having a semipermeable membrane is hung using a hanger, for example, if a plurality of culture dishes are placed in one well of a 6-well plate, they collide with each other, and may not function in a cell assay. In the present embodiment, for example, a plurality of culture dishes can be stably placed in one well of the 6-well plate.

FIG. 5(b) is a side view of the leg part 113. As shown in FIG. 5(b), in order to protect the contact surface with the leg part 113, the leg part 113 may be a round leg.

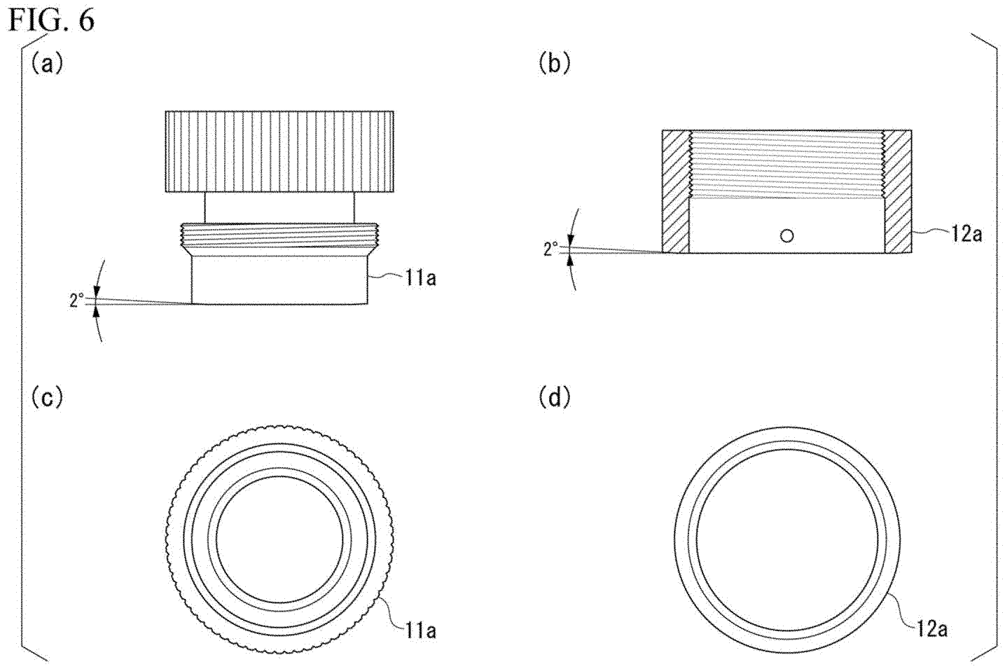

In addition, when a semipermeable membrane such as a vitrigel membrane is used for the first membrane 111 and the second membrane 121, as shown in FIG. 6, a surface 11a of the first culture dish 11 and a surface 12a of the second culture dish 12 preferably have a tapered structure (refer to FIGS. 6(a) and 6(b)). When the tapered structure is provided, the membrane is less likely to bend and an adhesive is less likely to protrude. In addition, preferably, a marking line is applied to the surface 11a of the first culture dish 11 and the surface 12a of the second culture dish 12 so that an adhesive does not protrude onto the membrane of the first membrane 111 and the second membrane 121 (refer to FIGS. 6(c) and 6(d)).

The tilt angle of the taper is preferably 10° or less, more preferably 5° or less, still more preferably 1° or more and 3° or less, and particularly preferably 2°. The marking line may be applied at a position at which the inner circumferential side and the outer circumferential side surfaces of the first and second culture dishes can be distinguished, and is preferably at a position at which the inner circumferential side and the outer circumferential side can be distinguished at 1:1, and more preferably at a position at which the inner circumferential side and the outer circumferential side can be distinguished at 1:2.

In addition, the cell culture device 1 of the present embodiment may further have an Nth culture dish (N is an integer of 3 or more), the Nth culture dish has an Nth membrane on the bottom surface, and the (N−1)th culture dish and the Nth culture dish may be mounted with a gap of which a height is adjustable therebetween. For example, as shown in FIG. 2(c), a third culture dish 16 is additionally provided, the third culture dish 16 has a third membrane on the bottom surface, and the second culture dish 12 and the third culture dish 16 may be mounted with a gap of which a height is adjustable therebetween. In this case, for example, the second culture dish 12 has a female screw cut on the inner circumferential surface, and the third culture dish 16 has a male screw cut on the outer circumferential surface. When the Nth culture dish (N is an integer of 3 or more) and the (N−1)th culture dish have such a relationship, the culture dishes can be mounted in tandem. Such a cell culture device is suitably used for an organ type chip to be described below.

### [Method of Producing Culture Dish]

A method of producing a culture dish to which a dried vitrigel membrane product is bonded according to the present embodiment is a method including a process 1 in which a sol is injected into recesses of a pedestal having one or more recesses, and a central part composed of a first material having low adsorptivity with respect to a hydrogel and a peripheral part composed of a second material having high adsorptivity with respect to a hydrogel on the bottom surface of the recess, and the sol is gelled, a process 2 in which the hydrogel obtained in the process 1 that is formed in the pedestal is dried and vitrified, a process 3 in which the dried hydrogel product obtained in the process 2 that is formed in the pedestal is hydrated, a process 4 in which the vitrigel obtained in the process 3 that is formed in the pedestal is dried and vitrified again, a process 5 in which a part of the dried vitrigel product obtained in the process 4 that slightly covers the top surface of the pedestal is cut off, and a process 6 in which a cylindrical member having an adhesive layer on the peripheral part on the side in contact with the dried vitrigel membrane product is placed in the recess of the pedestal, and the cylindrical member to which the dried vitrigel membrane product is bonded is then extracted from the pedestal, in this order.

### [Pedestal]

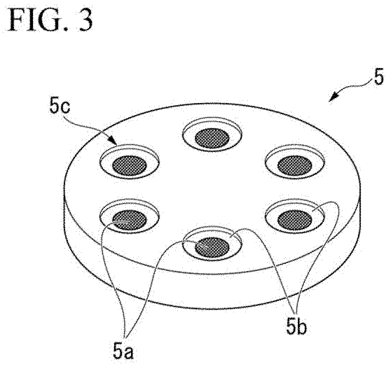

FIG. 3 is a perspective view of a pedestal 5. The pedestal 5 includes one or more recesses 5c. A dried vitrigel membrane product is produced in the recess 5c.

The recess 5c of the pedestal 5 has a smooth bottom surface, and side surfaces and the bottom surface that are perpendicular to each other because a dried vitrigel membrane product having a smooth surface can be obtained.

In addition, the cross-sectional area of the recess 5c can be set to a size so that the dried vitrigel membrane product has a desired size, and is not particularly limited. Specifically, the cross-sectional area of the recess 5c can be, for example, 4 mm² or more and 400 cm² or less, for example, 20 mm² or more and 40 cm² or less, and for example, 80 mm² or more and 4 cm² or less.

In addition, in the pedestal, the depth of the recess 5c can be appropriately adjusted so that the thickness of the dried vitrigel membrane product is a desired thickness, and the depth is preferably 1 μm or more and 5 mm or less, more preferably 5 μm or more and 3 mm or less, still more preferably 10 μm or more and 2 mm or less, and particularly preferably 20 μm or more and 1 mm or less.

In addition, in the pedestal 5, the cross-sectional shape of the recess 5c can be appropriately adjusted so that the shape of the dried vitrigel membrane product is a desired shape, and examples thereof include polygons such as a triangle, a rectangle (including a square, a rectangle, and a trapezoid), a pentagon, a hexagon, a heptagon, and an octagon; a circle, an ellipse, substantially a circle, an ellipse, substantially an ellipse, a semicircle, and a fan shape, but the present invention is not limited thereto. Among these, the cross-sectional shape of the recess 5c is preferably a circle.

In addition, in the pedestal 5, when the cross section of the recess 5c is circular, the diameter thereof can be appropriately adjusted so that the diameter of the dried vitrigel membrane product is a desired diameter, and can be, for example, 2 mm or more and 226 mm or less, for example, 5 mm or more and 72 mm or less, and for example, 10 mm or more and 23 mm or less.

In addition, regarding the material constituting the pedestal 5, on the bottom surface of the recess 5c, a central part 5a is composed of a first material having low adsorptivity with respect to a hydrogel, and a peripheral part 5b is composed of a second material having high adsorptivity with respect to a hydrogel. In addition, all parts other than the central part 5a on the bottom surface of the recess 5c may be composed of a second material.

Here, “central part 5a on the bottom surface of the recess 5c” means a position up to, for example, 9/10, preferably 4/5, more preferably 3/4, still more preferably 2/3, and particularly preferably 1/2 of the distance from the center of the bottom surface to the shortest edge. In addition, “the peripheral part 5b on the bottom surface of the recess 5c” means a part surrounding the central part on the bottom surface of the recess 5c.

In this specification, the “material having low adsorptivity with respect to a hydrogel” is a material that does not adsorb a hydrogel at all or a material that adsorbs a hydrogel with a weak force that allows desorption.

In addition, in this specification, the “material having high adsorptivity with respect to a hydrogel” is a material that completely adsorbs a hydrogel or a material that adsorbs a hydrogel with a strong force that does not allow desorption.

In addition, when the hydrogel is a gel containing proteins such as collagen, the material having low adsorptivity with respect to a hydrogel may be a material having many hydrophilic groups on the surface, and the material having high adsorptivity with respect to a hydrogel may be a material having many hydrophobic groups on the surface. The number of hydrophilic groups and hydrophobic groups on the surface can be appropriately adjusted according to the type of the hydrogel containing proteins to be used.

Examples of hydrophilic groups include a phosphorylcholine group and an alkylene glycol group.

Examples of hydrophobic groups include linear, branched and cyclic alkyl groups. The number of carbon atoms of the alkyl group is, for example, 1 or more and 20 or less, and for example, 4 or more and 20 or less.

Specific examples of first materials include stainless steel and poly(vinyl chloride), but the present invention is not limited thereto.

In addition, the first material may be, for example, a film on which a release agent such as silicone is laminated. When the dried vitrigel membrane product is produced so that the dried vitrigel membrane product and the surface on which a release agent layer of the film is laminated are in contact with each other, it is possible to easily peel off the dried vitrigel membrane product. Examples of materials of the film include polyethylene, polyethylene terephthalate, polystyrene, and polypropylene, and there is no particular limitation.

In addition, the first material may be, for example, an oil coating formed by applying an oil such as silicone to the bottom surface of the pedestal.

Specific examples of second materials include glass materials, polyacrylate (acrylic resin), polystyrene, and nylon, but the present invention is not limited thereto.

More specific examples of glass materials include soda-lime glass, Pyrex (registered trademark) glass, Vycor (registered trademark) glass, and quartz glass.

More specific examples of polyacrylate (acrylic resin) include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In addition, all parts other than the central part on the bottom surface of the recess may be composed of the second material exemplified above.

In addition, the first material may be detachably placed on the bottom surface of the recess of the pedestal. In this case, the first material is preferably bonded to the material constituting the bottom surface of the recess of the pedestal by physical peeling simply with a weak force that allows desorption. Specifically, the first material may be bonded to the material constituting the bottom surface of the recess of the pedestal via a salt such as PBS by physical peeling with tweezers or the like simply with a weak force that allows desorption. Alternatively, the first material may be bonded to the material constituting the bottom surface of the recess of the pedestal via a release agent layer containing a release agent such as silicone by physical peeling with tweezers or the like simply with a weak force that allows desorption.

In this case, examples of materials constituting the bottom surface of the recess of the pedestal present under the first material include the same materials as those of the above second material.

### <Process 1>

First, a sol is injected into the recess of the pedestal, and the sol is gelled.

The temperature at which the sol is kept warm can be appropriately adjusted according to the type of the sol to be used. For example, when the sol is a collagen sol, for heat retention during gelation, the temperature may be lower than the denaturation temperature of collagen depending on the animal species of collagen used, and generally, the temperature is kept at 20° C. or higher and 37° C. or lower, and thus gelation can be performed for several minutes to several hours.

### <Process 2>

Next, the obtained hydrogel that is formed in the pedestal is dried and vitrified.

When the hydrogel is dried, free water in the hydrogel can be completely removed, and additionally, bound water can be partially removed.

As the time period of the vitrification process (the process of proceeding with the partial removal of bound water after free water in the hydrogel is completely removed) is longer, vitrigel having excellent transparency and strength can be obtained when rehydrated. Here, as necessary, vitrigel obtained by rehydrating after vitrification for a short period can be washed with PBS or the like, and vitrified again.

### <Process 3>

Next, the obtained dried hydrogel product that is formed in the pedestal is hydrated. In this case, the product can be hydrated using saline, phosphate buffered saline (PBS) or the like.

### <Process 4>

Next, the obtained vitrigel that is formed in the pedestal is dried and vitrified again.

### <Process 5>

Next, in the obtained dried vitrigel product, for example, using a cylindrical blade (thin blade), a part of the dried vitrigel product that slightly covers the top surface of the pedestal is cut off. The cross section of the cylindrical blade can be slightly larger than the cross section of the recess of the pedestal. Specifically, the cross-sectional area of the cylindrical blade is preferably 1 times or more and 1.15 times or less, more preferably 1 times or more and 1.1 times or less, still more preferably 1 times or more and 1.07 times or less, and particularly preferably 1 times or more and 1.05 times or less the cross-sectional area of the recess of the pedestal.

In addition, the cross-sectional shape of the cylindrical blade can be the same as that of the cross section of the recess of the pedestal, and examples thereof include polygons such as a triangle, a rectangle (including a square, a rectangle, and a trapezoid), a pentagon, a hexagon, a heptagon, and an octagon; a circle, an ellipse, substantially a circle, an ellipse, substantially an ellipse, a semicircle, and a fan shape, but the present invention is not limited thereto. Among these, the cross-sectional shape of the through-hole is preferably circular.

In addition, when the cross section of the cylindrical blade is circular, the diameter of the cylindrical blade can be substantially the same as the diameter of the recess of the pedestal, and can be, for example, 2 mm or more and 226 mm or less, for example, 5 mm or more and 72 mm or less, and for example, 10 mm or more and 23 mm or less.

### <Process 6>

Next, a cylindrical member having an adhesive layer on the peripheral part on the side in contact with the dried vitrigel membrane product is placed in in the recess of the pedestal, and the cylindrical member to which the dried vitrigel membrane product is bonded is then extracted from the pedestal.

Regarding the adhesive constituting the adhesive layer, an adhesive having no cytotoxicity can be used, and a synthetic compound adhesive may be used or a natural compound adhesive may be used. Examples of synthetic compound adhesives include a urethane-based adhesive, a cyanoacrylate-based adhesive, a polymethylmethacrylate (PMMA), a calcium phosphate-based adhesive, and a resin-based cement. Examples of natural compound adhesives include fibrin glue and gelatin glue.

In addition, the adhesive layer may be composed of a double-sided tape. Regarding the double-sided tape, a tape having no cytotoxicity can be used, and a tape used for medical purposes or the like is suitably used. Specifically, for example, it has a structure in which a pressure-sensitive adhesive layer is laminated on both surfaces of a support, and examples thereof include those in which the pressure-sensitive adhesive layer is made of a known pressure-sensitive adhesive such as rubber-based, acrylic-based, urethane-based, silicone-based, and vinyl ether-based adhesives. More specific examples thereof include double-sided tapes for skin application (Product No: 1510, 1504XL, 1524, etc. commercially available from 3M Japan), double-sided adhesive tapes for skin (Product No: ST502, ST534, etc. commercially available from Nitto Denko Corporation), medical double-sided tapes (Product No: #1088, #1022, #1010, #809SP, #414125, #1010R, #1088R, #8810R, #2110R, etc. commercially available from Nichiban Medical Corp.), and thin foam base material double-sided adhesive tapes (Product No: #84010, #84015, #84020, etc. commercially available from DIC).

The cylindrical member used in the process 6 does not have the first membrane or the second membrane in the first culture dish or the second culture dish in [Cell culture device]. The adhesive layer is prepared on the outer circumferential side of the marking line applied to the surface of the first culture dish or the second culture dish.

Through the processes 1 to 6, a culture dish having a membrane is produced.

### [Method of Using Cell Culture Device]

As will be described below, the cell culture device of the present embodiment can be used for cell transport, a tissue type chip, an organ type chip, and an organ type chip system in addition to cell culture.

In this specification, “tissue” refers to a structural unit assembled in a pattern based on a certain lineage in which one type of stem cells differentiate, and has one role as a whole. For example, epidermal keratinocytes exhibit a barrier function as epidermis by differentiating stem cells present in an epidermis basal layer into cells constituting a granular layer via the stratum spinosum, and terminally differentiating them to form the stratum corneum. Therefore, the tissue type chip of the present embodiment can reproduce, for example, epithelial tissues, connective tissues, muscle tissues, or nerve tissues, by constructing a multicellular structure containing one type of cells derived from one cell lineage.

In addition, in this specification, an “organ” is composed of two or more types of tissues and has one function as a whole. Therefore, the organ type chip of the present embodiment can reproduce, for example, stomach, intestines, liver, kidney or the like by constructing a multicellular structure containing at least two types of cells having different cell lineages.

In addition, in this specification, the “organ system” is a group of two or more organs having the same function or a group of two or more organs having a series of functions as a whole. Therefore, the organ type chip system of the present embodiment can reproduce, for example, the organ system such as the digestive system, the circulatory system, the respiratory system, the urinary system, the reproductive system, the endocrine system, the sensory system, the nervous system, the musculoskeletal system, and the nervous system, by combining a plurality of tissue type chips or organ type chips. Here, a living body maintains homeostasis according to the interaction of these organ systems. In the organ type chip system of the present embodiment, since a plurality of organ type chips having different organ systems can be combined, it is also possible to analyze the interaction between organs having different organ systems. For example, in an organ type chip system in which a small intestine type chip, a liver type chip, and a nerve type chip are connected in this order, when a drug is added to the small intestine type chip, the drug absorbed by the small intestine type chip is metabolized in the liver type chip, and it is possible to analyze the toxicity of the hepatic metabolites of the drug excreted from the liver type chip on the nerve type chip.

### [Cell Culture Method]

A cell culture method of the present embodiment is a method using the above cell culture device.

According to the culture method of the present embodiment, by using the mounting mechanism of the cell culture device, for example, the first culture dish and the second culture dish are mounted by screwing, and thus cells can be easily cultured, and a multicellular structure can be constructed. In addition, cells can be maintained for about 3 to 30 days, and cells can be maintained for a longer period than in the related art. In addition, according to the culture method of the present embodiment, it is possible to obtain a tissue type chip to be described below.

In the culture method of the present embodiment, details will be described below.

First, a culture medium in which cells are suspended is prepared. Next, the suspension is injected into the first culture dish. When the first culture dish has a hole at the gap with the height required for cell encapsulation, the hole may be blocked in advance with an aseptic tape or the like. When the hole is blocked if the seeded cells are point-bonded or surface-bonded to the first membrane, the tape or the like is peeled off to open the hole, the second culture dish is mounted, and an excess culture solution is discharged from the hole.

Next, the cells in the cell culture device may be cultured in a gas phase and/or liquid phase to construct a multicellular structure. Mammalian cells are cultured in a humidifying incubator at a temperature of 37° C. in the presence of 5% CO₂. Therefore, for culturing in a gas phase, for example, a cell culture device in which cells are seeded may be placed in a container such as an empty petri dish, and the culture medium in the cell culture device may be replaced at a time when cell nutrients are not depleted. When cells are seeded in the second culture dish having a semipermeable membrane such as vitrigel, the second culture dish is pulled up from the first culture dish to form a gap, and a first culture dish having no liquid can be brought into a gas phase.

In addition, culturing in a liquid phase may be performed using, for example, a container such as a multi-well plate containing a culture medium. When cells are seeded in a second culture dish having a semipermeable membrane such as vitrigel, the second culture dish is pulled up from the first culture dish to form a gap, and the first culture dish filled with a culture medium can be brought into a liquid phase.

Examples of cells used in the culture method of the present embodiment include vertebrate cells such as mammalian cells, avian cells, reptilian cells, amphibian cells, and fish cells; invertebrate cells such as insect cells, crustacean cells, mollusc cells, and protozoan cells; bacteria such as gram-positive bacteria (for example, bacillus species, etc.), and gram-negative bacteria (for example, Escherichia coli, etc.); yeasts; plant cells; and small living individuals composed of single cells or multiple cells.

Examples of small living individuals include unicellular organisms such as amoeba, paramecium, closterium, diatoms, chlorella, euglena, and phacus; micro crustacean animals such as daphnia, artemia larvas, copepoda, ostracodi, thecostraca larvas, phyllocarida larvas, peracarida larvas, and eucarida larvas; planarian flatworms (including regenerated planarian after fine cutting), terrestrial arthropod larvas, Nematoda, plant seeds (particularly, germinated seed), callus, protoplast, marine microorganisms (for example, marine bacteria such as Vibrio, Pseudomonas, Aeromonas, Alteromonas, Flavobacterium, Cytophaga, and Flexibacter, and algae such as blue algae, crypto algae, dinoflagellate, diatom, raphidophytes, golden algae, haptophytes, euglenophyte, prasinophyceae, green algae, etc.), larval fish, and juvenile shellfish, but the present invention is not limited thereto.

For example, when germinated seeds are cultured using the cell culture device of the present embodiment, a cell culture device made of a biodegradable material is used, and the membrane on the top surface has a hardness at which the germinated buds can still penetrate, and thus the germinated seeds put into the device can be directly planted in soil to grow plants.

Here, in this specification, “biodegradable material” is a material having a property of being decomposed into an inorganic substance by microorganisms in soil or water.

Examples of vertebrate cells (particularly, mammalian cells) include germ cells (sperm, egg, etc.), somatic cells constituting a living body, stein cells, progenitor cells, cancer cells isolated from a living body, cells that are isolated from a living body, acquire immortalization ability, and are stably maintained outside the body (cell lines), cells that are isolated from a living body and artificially genetically modified, and cells that are isolated from a living body and of which nuclei are artificially exchanged, but the present invention is not limited thereto. In addition, a multicellular spherical aggregate (spheroid) of these cells may be used. In addition, small tissue pieces isolated from normal tissues or cancer tissues of a living body may be used without change in the same manner as the cell mass.

Examples of somatic cells that constitute a living body include cells collected from arbitrary tissues such as skin, kidneys, spleen, adrenal glands, livers, lungs, ovaries, pancreas, uterus, stomach, colon, small intestine, large intestine, bladder, prostate, testis, thymus, muscles, connective tissues, bones, cartilage, vascular tissues, blood, heart, eyes, brain, and nervous tissues, but the present invention is not limited thereto. More specific examples of somatic cells include fibroblasts, bone marrow cells, immune cells (for example, B lymphocytes, T lymphocytes, neutrophils, macrophages, monocytes, etc.), red blood cells, thrombocytes, osteocytes, bone marrow cells, pericytes, dendritic cells, epidermal keratinocytes, adipocytes, mesenchymal cells, epithelial cells, epidermal cells, endothelial cells, vascular endothelial cells, lymphatic endothelial cells, hepatocytes, pancreatic islet cells (for example, α cells, β cells, δ cells, ε cells, PP cells, etc.), chondrocytes, cumulus cells, glial cells, nerve cells (neurons), oligodendrocytes, microglia, astrocytes, myocardial cells, esophageal cells, muscle cells (for example, smooth muscle cells, skeletal muscle cells, etc.), melanocytes, and mononuclear cells, but the present invention is not limited thereto.

Stem cells are cells that have an ability to replicate themselves and an ability to differentiate into other multiple lineages of cells in combination. Examples of stem cells include embryonic stem cells (ES cells), embryonic tumor cells, embryonic reproductive stem cells, induced pluripotent stem cells (iPS cells), nerve stem cells, hematopoietic stem cells, mesenchymal stem cells, hepatic stem cells, pancreatic stem cells, muscle stem cells, germline stem cells, intestinal stem cells, cancer stem cells, and hair follicle stem cells, but the present invention is not limited thereto.

Progenitor cells are cells in the process of differentiating from stem cells into specific somatic cells or germ cells.

Cancer cells are cells derived from somatic cells and have acquired an infinite proliferation ability, and are malignant neoplasms that invade surrounding tissues or cause metastasis. Examples of cancers derived from cancer cells include breast cancer (for example, invasive ductal carcinoma in situ, ductal carcinoma in situ, inflammatory breast cancer, etc.), prostate cancer (for example, hormones-dependent prostate cancer, hormone-independent prostate cancer, etc.), pancreatic cancer (for example, pancreatic ductal cancer, etc.), stomach cancer (for example, papillary adenocarcinoma, mucinous adenocarcinoma, adenosquamous carcinoma, etc.), lung cancer (e.g., non-small cell lung cancer, small cell lung cancer, malignant mesoderm, etc.), colon cancer (e.g., gastrointestinal stromal tumor, etc.), rectal cancer (for example, gastrointestinal stromal tumor, etc.), colorectal cancer (for example, familial colorectal cancer, hereditary nonpolyposis colorectal cancer, gastrointestinal stromal tumor, etc.), small intestine cancer (e.g., non-Hodgkin's lymphoma, gastrointestinal stromal tumor, etc.), esophageal cancer, duodenal cancer, tongue cancer, pharyngeal cancer (for example, nasopharyngeal cancer, oropharyngeal cancer, hypopharyngeal cancer, etc.), head and neck cancer, salivary gland cancer, brain tumor (for example, pineal astrocytoma, pilocytic astrocytoma, diffuse astrocytoma, anaplastic astrocytoma, etc.), neurinoma, liver cancer (for example, primary liver cancer, extrahepatic cholangiocarcinoma, etc.), kidney cancer (for example, renal cell carcinoma, transitional cell carcinoma of the renal pelvis and urinary tract, etc.), gallbladder cancer, pancreatic cancer, endometrial cancer, cervical cancer, ovarian cancer (for example, epithelial ovarian cancer, extragonadal germ cell tumor, ovarian germ cell tumor, ovarian low malignant potential tumor, etc.), bladder cancer, urethral cancer, skin cancer (for example, intraocular (eye) melanoma, Merkel cell carcinoma, etc.), hemangiomas, malignant lymphomas (for example, reticular sarcoma, lymph sarcoma, Hodgkin's disease, etc.), melanoma (malignant melanoma), thyroid cancer (for example, medullary thyroid cancer, etc.), parathyroid cancer, nasal cavity cancer, sinus cancer, bone tumor (for example, osteosarcoma, Ewing tumor, uterine sarcoma, soft tissue sarcoma, etc.), metastatic medulloblastoma, angiofibroma, elevated cutaneous fibrosarcoma, retinal sarcoma, penile cancer, testicular cancer, pediatric solid cancer (for example, Wilms' tumor, pediatric renal tumor, etc.), Kaposi's sarcoma, Kaposi's sarcoma caused by AIDS, maxillary sinus tumor, fibrous histiocytoma, leiomyosarcoma, rhabdomyosarcoma, chronic myeloproliferative disorder, and leukemia (for example, acute myeloid leukemia, acute lymphoblastic leukemia, etc.), but the present invention is not limited thereto.

In addition, in this specification, “tumor” is used to represent a diagnosis name, and “cancer” is used to represent a general term for malignant neoplasms.

A cell line is a cell that has acquired an infinite proliferation ability according to an artificial operation in vitro. Examples of cell lines include HCT116, Huh7, and HEK293 (human fetal kidney cell), HeLa (human cervical cancer cell line), HepG2 (human liver cancer cell line), UT7/TPO (human leukemia cell line), CHO (Chinese hamster ovary cell line), MDCK, MDBK, BHK, C-33A, HT-29, AE-1, 3D9, Ns0/1, Jurkat, NIH3T3, PC12, S2, Sf9, Sf21, High Five, and Vero, but the present invention is not limited thereto.

The culture medium of animal cells used in the culture method of the present embodiment may be a basal culture medium containing components required for cell survival and proliferation (inorganic salts, carbohydrate, hormones, essential amino acids, non-essential amino acids, and vitamin), and can be appropriately selected according to the type of cells. Examples of culture media include Dulbecco's modified Eagle's medium (DMEM), Minimum Essential Medium (MEM), RPMI-1640, Basal Medium Eagle (BME), Dulbecco's Modified Eagle's Medium: Nutrient Mixture F-12(DMEM/F-12), and Glasgow Minimum Essential Medium (Glasgow MEM), but the present invention is not limited thereto.

In addition, for a culture medium of bacteria, yeast, plant cells, and small living individuals composed of single cells or multiple cells, a culture medium having a composition suitable for each growth may be prepared.

In addition, in the culture method of the present embodiment, an extracellular matrix-derived component, a physiologically active substance, and the like may be mixed and injected into a culture medium in which cells are suspended.

Examples of extracellular matrix-derived components include those exemplified for the above “porous membrane.”

In addition, examples of physiologically active substances include cell growth factors, inductive differentiating factors, and cell adhesion factors, but the present invention is not limited thereto. For example, when cells to be injected are stem cells or progenitor cells, due to inclusion of an inductive differentiating factor, it is possible to induce differentiation of the stem cells or progenitor cells and construct a multicellular structure in which desired tissues are reproduced.

In the culture method of the present embodiment, animal cell culture conditions can be appropriately selected according to the type of cells to be cultured. The culture temperature may be, for example, 25° C. or higher and 40° C. or lower, for example, 30° C. or higher and 39° C. or lower, and for example, 35° C. or higher and 39° C. or lower. In addition, the culture environment may be, for example, a humidifying incubator in which the culture medium does not dry under about a 5% CO₂ condition.

The culture time can be appropriately selected according to the type of cells, the number of cells, or the like, and may be, for example, 3 days or longer and 30 days or shorter, for example, 5 days or longer and 20 days or shorter, and for example, 7 days or longer and 15 days or shorter.

In addition, in culture conditions for bacteria, yeast, plant cells, and small living individuals composed of single cells or multiple cells, an environment and a time suitable for each growth may be set.

### [Cell Transport Method]

A cell transport method of the present embodiment is a method using the above cell culture device. According to the transport method of the present embodiment, cells can be transported safely, reliably, and easily, and can be transported for a long period.

The transport method of the present embodiment will be described below in detail.

First, a culture medium in which cells are suspended is prepared. Next, the culture medium in which cells are suspended is injected into the above first culture dish.

In this case, when both the first membrane and the second membrane are a PET film, preferably, the first culture dish is filled with the culture medium and is covered with the second culture dish.

When the first membrane is a PET film and the second membrane is a sterilizing filter, preferably, the first culture dish and the second culture dish are filled with the culture medium, and the second culture dish is covered with a lid.

When the first membrane is a vitrigel membrane and the second membrane is a dialysis membrane, preferably, the first culture dish and the second culture dish are filled with the culture medium, the first culture dish is covered with the protection part, and the second culture dish is covered with a lid (refer to FIG. 2(d)). In addition, the gap between the first culture dish and the protection part may be filled with the culture medium. With such a structure, it is possible to transport the cell culture device alone.

Examples of cells or small living individuals used in the transport method of the present embodiment include those exemplified in the above “cell culture method.” In addition, examples of culture media used in the transport method of the present embodiment include those exemplified in the above “cell culture method.”

The cells encapsulated in the cell culture device may be in the process of constructing the multicellular structure or after the multicellular structure may have already been constructed. With regard to these, since it can be immediately used for an in vitro test system or living body transplantation, in the transport method of the present embodiment, cells are preferably encapsulated in the cell culture device after the multicellular structure has been constructed.

Conditions for transport can be appropriately selected according to the type of cells or small living individuals to be transported.

The temperature during transportation may be, for example, 4° C. or higher and 40° C. or lower, for example, 10° C. or higher and 39° C. or lower, and for example, 18° C. or higher and 37° C. or lower.

In addition, in the transport environment, when cells are animal cells, a cell culture device in which the cells are encapsulated may be encapsulated in a sealed container filled with the culture medium to the full volume without being covered with the lid and the protection part. Alternatively, a cell culture device in which the cells are encapsulated may be encapsulated in a sealed container filled with the culture medium to a part of the volume without being covered with the lid and the protection part, and may be, for example, under a condition of air containing about 5% CO₂ in a gas part in the sealed container.

In addition, the cell culture device composed of the first culture dish and the second culture dish alone may be in a state in which cells are cultured in the first culture dish, the culture medium is filled to the full volume, and the protection part covering the first membrane is provided, a state in which cells are cultured in the second culture dish, the culture medium is filled to the full volume, and the second culture dish is closed with the lid, or in a state in which both the protection part and the lid are provided. Here, the gap between the protection part that covers the first membrane and the first membrane can be filled with the culture medium.

The transportation time can be appropriately selected according to the type of cells, the number of cells, or the like, and may be, for example, 1 hour or longer and 30 days or shorter, for example, 12 hours or longer and 7 days or shorter, and for example, 1 day or longer and 3 days or shorter.

### [Tissue Type Chip]

A tissue type chip of the present embodiment includes the above cell culture device having one type of cells (particularly, animal cells) in at least the first culture dish.

The tissue type chip of the present embodiment does not require construction of a culture model from scratch, and as an alternative to the conventional culture model or animal experiments, it can be used for screening of candidate drugs for various diseases or for an evaluation test system of dynamics and toxicity of chemical substances including candidate drugs on normal tissues.

In addition, the conventional culture model or regenerated tissues for transplantation should be immediately used after construction, which is time-constrained, but the tissue type chip of the present embodiment can be cultured for a long period.

The density of cells encapsulated in the tissue type chip of the present embodiment varies depending on the type of tissues to be constructed and is preferably 2.0×10³ cells/mL or more 1.0×10⁹ cells/mL, or less, and more preferably 2.0×10⁵ cells/mL or more and 1.0×10⁷ cells/mL, or less.

When the cell density is within the above range, a tissue type chip having a cell density closer to that of living tissues can be obtained.

The tissue type chip of the present embodiment can be produced using the method described in the above “cell culture method.” In addition, maintenance conditions for the tissue type chip after production may be the same conditions as the culture conditions described in the above “cell culture method.” In addition, the inside the tissue type chip may contain a culture medium or a gas such as air, and may not contain a culture medium or a gas such as air. When the tissue type chip does not contain a culture medium or a gas such as air, cells or cells and extracellular matrix-derived components are closely bonded to construct a multicellular structure having a configuration closer to that of tissues in a living body.

### [Organ Type Chip]

The organ type chip of the present embodiment includes a cell culture device having a culture dish containing different types of cells.

The organ type chip of the present embodiment does not require construction of a culture model from scratch, and as an alternative to the conventional culture model or animal experiments, it can be used for screening of candidate drugs for various diseases or for an evaluation test system of dynamics and toxicity of chemical substances including candidate drugs on normal organs.

In addition, the conventional culture model or regenerated tissues for transplantation should be immediately used after construction, which is time-constrained, but the organ type chip of the present embodiment can be cultured for a long period.

Examples of cells encapsulated in the organ type chip of the present embodiment include those exemplified in the above “cell culture method.” In addition, regarding the type of encapsulated cells, at least two types of cells may be encapsulated, and the type may be appropriately selected according to the type of organ to be constructed.

In addition, the cells encapsulated in the organ type chip of the present embodiment may be in the process of constructing the multicellular structure or after the multicellular structure may have already been constructed. In the organ type chip of the present embodiment, even after the encapsulated cells construct a multicellular structure, they can be cultured for a long period of about 3 to 21 days.

In addition, for example, in the organ type chip of the present embodiment, a multicellular structure (that is, epithelial tissues) composed of epithelial cells (for example, epidermal keratinocytes) is encapsulated in the second culture dish of the cell culture device, a multicellular structure (that is, mesenchyme tissues) composed of mesenchymal cells (for example, dermal fibroblasts) is encapsulated in the first culture dish, and thus the transfer of substances between tissues can be easily reproduced in the cell culture device.

The density of cells encapsulated in the organ type chip of the present embodiment, the culture method, and the like are the same as those in the “tissue type chip.”

The organ type chip of the present embodiment alone can reproduce organs, for example, liver, stomach, and intestines. In addition, when a plurality of organ type chips of the present embodiment are combined, it is possible to reproduce the organ system, for example, the digestive system, the circulatory system, the respiratory system, the urinary system, the reproductive system, the endocrine system, the sensory system, the nervous system, the musculoskeletal system, and the nervous system.

### [Organ Type Chip System]

The organ type chip system of the present embodiment includes at least two of the above tissue type chips or the above organ type chips, and the tissue type chips or the organ type chips are connected while maintaining cell encapsulation.

The organ type chip system of the present embodiment does not require construction of a culture model from scratch, and as an alternative to the conventional culture model or animal experiments, it can be expected to be used for screening of candidate drugs for various diseases or for an evaluation test of dynamics and toxicity of chemical substances including candidate drugs on a plurality of normal tissues and organs.

### [Hepatocyte Culture Device]

In one embodiment, the present invention provides a hepatocyte culture device that promotes accumulation and excretion of hepatic metabolites in a bile canaliculus-like structure, the device including: a first cell culture product containing a plurality of hepatocytes; a second culture dish which is able to construct a bile canaliculus-like structure between hepatocytes in the first cell culture product, and has a membrane that a physiologically active substance is able to permeate on the bottom surface and in which the first cell culture product is accommodated; a second cell culture product that is able to increase an excretion activity of hepatic metabolites in the first cell culture product; and a first culture dish in which the second cell culture product is accommodated, wherein the first cell culture product is placed on the second cell culture product and co-cultured, and wherein the first culture dish and the second culture dish are mounted with a gap of which a height is adjustable therebetween.

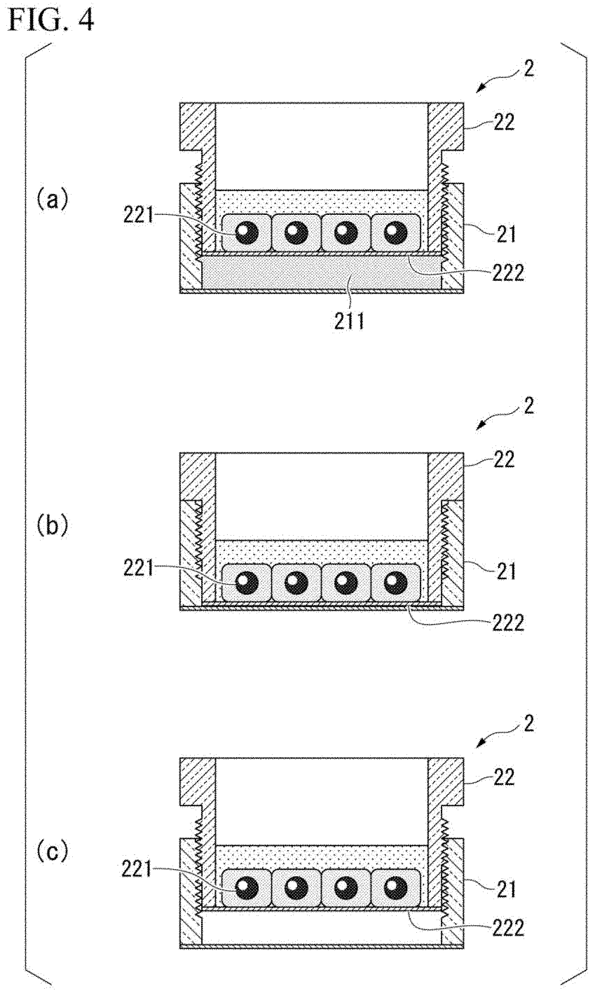

FIG. 4(a) is a side view of a hepatocyte culture device 2 of the present embodiment.

As shown in FIG. 4(a), in the hepatocyte culture device 2, a first culture dish 21 and a second culture dish 22 are mounted with a gap of which a height is adjustable therebetween. The second culture dish 22 accommodates a first cell culture product 221 containing a plurality of hepatocytes and has a membrane 222 that a physiologically active substance is able to permeate on the bottom surface. The first culture dish 21 accommodates a second cell culture product 211 that is able to increase an excretion activity of hepatic metabolites in the first cell culture product 221 in the gap.

In the hepatocyte culture device of the present embodiment, the first cell culture product contains a plurality of hepatocytes. The hepatocytes are preferably cultured hepatocytes derived from normal liver tissues, liver cancer tissues or stem cells (ES cells, iPS cells, mesenchymal stem cells, etc.) selected from the group consisting of humans, rats, monkeys, apes, cats, dogs, pigs, cows, sheep, horses, chickens and ducks, more preferably frozen human hepatocytes, still more preferably human liver tumor-derived cell lines, particularly preferably HepG2 cells, which are one of the human liver cancer-derived cell lines, and most preferably HepG2-NIAS cells (RCB4679 strain).

The HepG2 cells are cheaper than frozen human hepatocytes and HepaRG (registered trademark) cells, which are human liver tumor-derived cell lines, and can further increase the activity of CYP3A4 to about half of the differentiated HepaRG cells that express average activity of frozen human hepatocytes in a short period of 3 days according to rapid activation of liver functions to be described below.

In the hepatocyte culture device of the present embodiment, materials other than the membrane that a physiologically active substance is able to permeate are the same as in the [Cell culture device].

The membrane that a physiologically active substance is able to permeate is preferably the hydrogel described in the above [Cell culture device], and more preferably vitrigel. In the present invention, the “physiologically active substance” is a culture medium component and a substance produced from cultured cells, and includes a substance having a small molecular weight to a substance having a molecular weight of 20,000 or more. For example, various medicines including antibiotics, cell growth factors, inductive differentiating factors, cell adhesion factors, antibodies, enzymes, cytokines, hormones, lectin, or fibronectin, entactin, and osteopontin as extracellular matrix components that do not gel may be exemplified.

In the first cell culture product, an example of a method of constructing a bile canaliculus-like structure between hepatocytes will be described below. The first cell culture product to be used may be any of the above hepatocytes, but use of HepG2 cells will be exemplified.

First, in the hepatocyte culture device 2, the second culture dish 22 is positioned at the lowermost position, and a sample obtained by suspending the first cell culture product in the culture medium is then seeded in the second culture dish 22. Since the membrane 222 that a physiologically active substance is able to permeate is in contact with the bottom surface of the first culture dish 21, a “liquid phase-membrane-solid phase” state is formed. In this state, the cells are cultured until they become confluent. The culture time varies depending on the cells used, but for example, HepG2 cells are preferably cultured for about 2 days (refer to FIG. 4(b)).

In the present embodiment, the “culture medium” may be any medium used for culturing normal cells, and examples thereof include Dulbecco's Modified Eagle's Medium (DMEM), Minimum Essential Media (MEM), Iscove's Modified Dulbecco's Medium (IMDM), and Glasgow's Minimum Essential Medium (GMEM).

After the cells are in a confluent state, the second culture dish 22 is pulled up from the first culture dish 21 to form a gap. That is, the cells are additionally cultured in a “liquid phase-membrane-gas phase” state (refer to FIG. 4(c)). The culture time is preferably about 20 hours to 24 hours.

It generally takes 10 days to 14 days for HepaRG (registered trademark) cell lines or human iPS cell-derived hepatocytes to activate liver functions. However, when cells in a confluent state and cells in a “liquid phase-membrane-gas phase” state are cultured, for example, in HepG2 cells, liver functions are activated in a short period of 3 days.

In the present embodiment, “liver function” indicates an ability to synthesize albumin and urea and a pharmacokinetic-related function, for example, the activity of CYP3A4, and the ability to synthesize albumin and urea is activated to the same level as normal frozen human hepatocytes. In addition, the activity of CYP3A4 can be increased to about half of the differentiated HepaRG cells that express average activity of frozen human hepatocytes.

In the activated state of liver functions, bile canaliculus-like structures and tight junctions are constructed between cells. Generally, in hepatocytes, cells are bonded by tight junctions, and gaps formed between cells are connected in a tubular shape to form bile canaliculi, which are connected to the bile duct.

Although the details of the process in which hepatocytes are activated are unknown, it is speculated that, when oxygen in a gas phase is supplied through the membrane, cells favorably grow under an aerobic culture condition despite a high cell density, and as a result, a self-assembling ability of hepatocytes can be sufficiently elicited under culture conditions.

In the present embodiment, the second cell culture product may be normal cultured cell groups, feeder cell groups treated with mitomycin C, dead cell groups fixed with methanol or the like, or a conditioned culture medium of normal cultured cell groups as long as they can increase the excretion activity of hepatic metabolites in the first cell culture product. Examples thereof include cultured cells derived from epithelium, mesenchyme or endothelium selected from the group consisting of humans, rats, monkeys, apes, cats, dogs, pigs, cows, sheep, horses, chickens and ducks, and intrahepatic bile duct epithelial cells, extrahepatic bile duct epithelial cells, vascular endothelial cells, fibroblasts, and culture supernatants of these cells may be exemplified.

The excretion activity of the hepatic metabolites is an activity of excreting hepatic metabolites accumulated in bile canaliculus-like structures between hepatocytes and in the hepatocytes in the first cell culture product.

As an example of the second cell culture product, TFK-1 cells, which are one of the human bile duct cancer-derived cell lines, may be exemplified. For example, TFK-1 cells are accommodated in the first culture dish 21, and are co-cultured with HepG2 cells accommodated in the second culture dish 22.

In this case, compared to when co-culture with TFK-1 cells as the second cell culture product is not performed, a total amount of hepatic metabolites excreted (upper excretion amount+lower excretion amount) and a proportion of a lower excretion amount increase. This is because co-culturing with the second cell culture product promotes excretion of hepatic metabolites accumulated in bile canaliculus-like structures between hepatocytes and in the hepatocytes in the first cell culture product. When hepatic metabolites are excreted not only in bile but also in urine, excretion of hepatic metabolites occurs not only from bile canaliculus-like structures but also from the cytoplasm of hepatocytes.

Therefore, the first cell culture product is co-cultured with the second cell culture product, and thus the amount of excretion of metabolites from hepatocytes increases.

In the present embodiment, the time for which the first cell culture product is co-cultured with the second cell culture product is preferably 3 minutes or longer, and more preferably 30 minutes or longer. In addition, in consideration of the growth state of the culture product, the co-culture time is preferably within 9 days, and more preferably within 2 days.

In addition, the temperature condition for co-culturing is generally any temperature recommended for cell culture, and the lower limit value is preferably 35° C. or higher, and more preferably 37° C. or higher. The upper limit value is preferably 40° C. or lower and more preferably 37° C. or lower.

According to the hepatocyte culture device of the present embodiment, a substance metabolized by hepatocytes in an environment in which the inside of a living body is reflected can be easily obtained in a short time and inexpensively. Hepatic metabolites excreted in bile canaliculus-like structures constructed between hepatocytes can also be easily collected without destroying tight junctions between hepatocytes.

### [Human Corneal Epithelial Model]

In one embodiment, the present invention provides a human corneal epithelial model including a first cell culture product containing a plurality of human corneal epithelial cells; a first culture dish which has a membrane that a physiologically active substance is able to permeate on the bottom surface in the first cell culture product and in which the first cell culture product is accommodated; and a second culture dish having a sterile filtration membrane on the bottom surface, wherein the second culture dish is placed on the first culture dish, and wherein the first culture dish and the second culture dish are mounted with a gap of which a height is adjustable therebetween.

The human corneal epithelial cells are not particularly limited, and examples thereof include HCE-T strain (RCB2280 strain). Examples of membranes that are able to permeate a physiologically active substance include those exemplified in [Hepatocyte culture device].

In a method of producing a human corneal epithelial model of the present embodiment, first, a culture medium in which human corneal epithelial cells are suspended is injected into a first culture dish covered with a protection part filled with a culture medium, and after culture for 2 days, the culture medium in the first culture dish is removed, and the lower side of the first culture dish is in a liquid phase, and the upper side is in a gas phase. In addition, culturing is performed for about 4 days to complete the human corneal epithelial model.

Subsequently, the first culture dish is filled with the culture medium, the second culture dish is mounted, the second culture dish is then filled with the culture medium, and additionally, a lid is mounted on the second culture dish. The human corneal epithelial model constructed in the cell culture device in this manner with the lid and the protection part mounted is transported to a cosmetics company or the like. Upon arrival, the human corneal epithelial model can be immediately used in an evaluation system by simply removing the lid and the protection part.

### [Oxygen Partial Pressure Control Model]

In one embodiment, the present invention provides an oxygen partial pressure control model including an Mth culture dish (M is an integer of 2 or more) having an Mth membrane on the bottom surface, an (M−1)th culture dish having an (M−1)th membrane on the bottom surface, and an oxygen generation mechanism, and in which the Mth culture dish and the (M−1)th culture dish are mounted with a gap of which a height is adjustable therebetween or mounted via the oxygen generation mechanism.

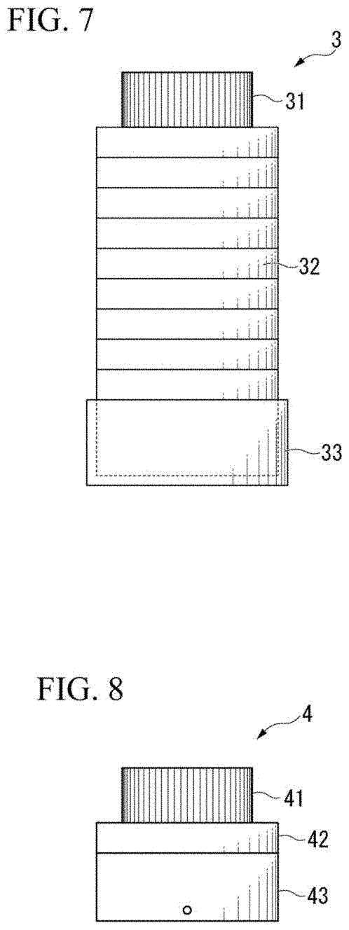

FIG. 7 is a side view of an oxygen partial pressure control model 3 of the present embodiment. In the oxygen partial pressure control model 3, M is 10, and 10 culture dishes are vertically stacked. In the present embodiment, a lid 31 and a protection part 33 are provided, and a sealed structure is formed. For example, when the oxygen partial pressure control model 3 has an oxygen generation mechanism 32, the oxygen partial pressure is highest in the vicinity of the oxygen generation mechanism 32, the oxygen partial pressure decreases in the vertical direction, and a gradient of the oxygen partial pressure can be formed.

The oxygen generation mechanism 32 is not particularly limited as long as it generates oxygen, and examples thereof include a culture dish containing a reagent that generates oxygen according to a chemical reaction.

### [Human Small Intestine Model]

In one embodiment, the present invention provides a human small intestine model which includes an anaerobic bacterial culture product; a second culture dish in which the anaerobic bacterial culture product having a sterile filtration membrane on the bottom surface is accommodated; a small intestine-derived cell culture product; a first culture dish which has a semipermeable membrane having liquid-tightness in a gas phase and semipermeability in a liquid phase on the bottom surface and in which the small intestine-derived cell culture product is accommodated; and an oxygen supply mechanism, wherein the anaerobic bacterial culture product is placed on the small intestine-derived cell culture product, the small intestine-derived cell culture product is placed on the oxygen supply mechanism, and the first culture dish and the second culture dish are mounted with a gap of which a height is adjustable therebetween.

FIG. 8 is a side view of a human small intestine model 4 of the present embodiment. A second culture dish 42 has a sterile filtration membrane on the bottom surface, and accommodates an anaerobic bacterial culture product. Examples of sterile filtration membranes include a sterilizing filter having a pore size of 0.22 μm. A first culture dish 43 has a semipermeable membrane having liquid-tightness in a gas phase and semipermeability in a liquid phase on the bottom surface, and accommodates a small intestine-derived cell culture product.

In the present embodiment, the human small intestine model 4 includes a lid 41, and the second culture dish 42 is under anaerobic conditions. Examples of anaerobic bacteria include Staphylococcus (gram-positive coccus), Corynebacterium (grain-positive bacillus), Listeria (grain-positive bacillus), and Escherichia coli (grain-negative bacillus), and Escherichia coli is preferable in consideration of reproduction of the human small intestine.

Cells derived from the small intestine in the first culture dish 43 are not particularly limited, and examples thereof include Caco2 cells. In culturing of cells derived from the small intestine, the first culture dish 43 is under aerobic conditions. The first culture dish 43 has an oxygen supply mechanism thereunder. The oxygen supply mechanism preferably includes a mechanism that uses oxygen in air or an oxygen generation mechanism. Since the first culture dish 43 may be under aerobic conditions, it may be an open system, and it may have a vitrigel membrane on the bottom surface and may be simply in an open state.

In recent years, it has been found that the imbalance of intestinal bacterial flora induces cancer and mental disorders. The apical side (lumen side) of intestinal epithelial cells is anaerobic due to the presence of bacterial flora, and the lower side of epithelial cells is aerobic because there are blood vessels and red blood cells carry oxygen. As described above, the second culture dish 42 is under anaerobic conditions, the first culture dish 43 is under aerobic conditions, and an intestinal environment is reproduced.

### EXAMPLES

While the present invention will be described below with reference to examples, the present invention is not limited to the following examples.

### Example 1

### <<Production of Cell Culture Device>>

### [Method of Producing Vitrigel Membrane]

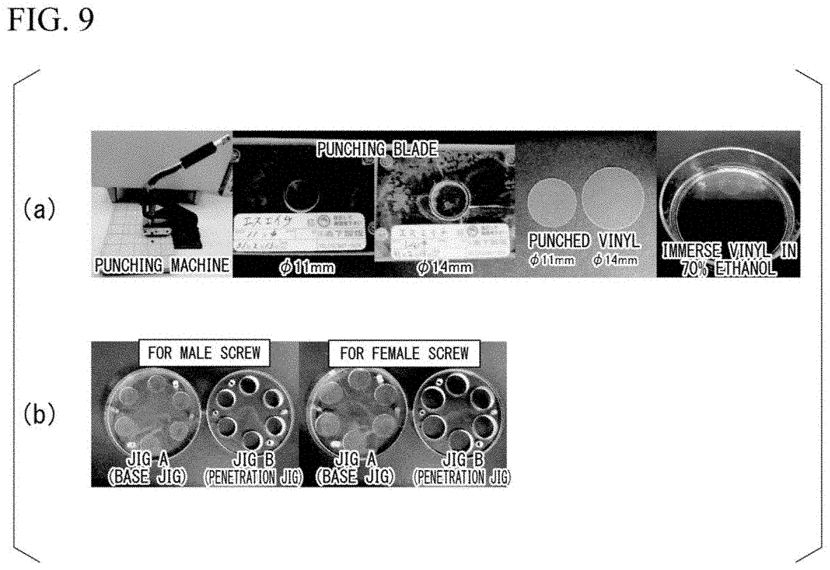

Vinyl (Unipack) was punched into φ11 mm and φ14 mm shapes with a punching machine (refer to FIG. 9(a)). The punched vinyl was placed in a φ60 mm petri dish containing 70% ethanol, immersed for 10 minutes, and sterilized and dried (refer to FIG. 9(a)).

70% ethanol was sprayed on an acrylic jig for sterilization and dried in a clean bench (refer to FIG. 9(b)).

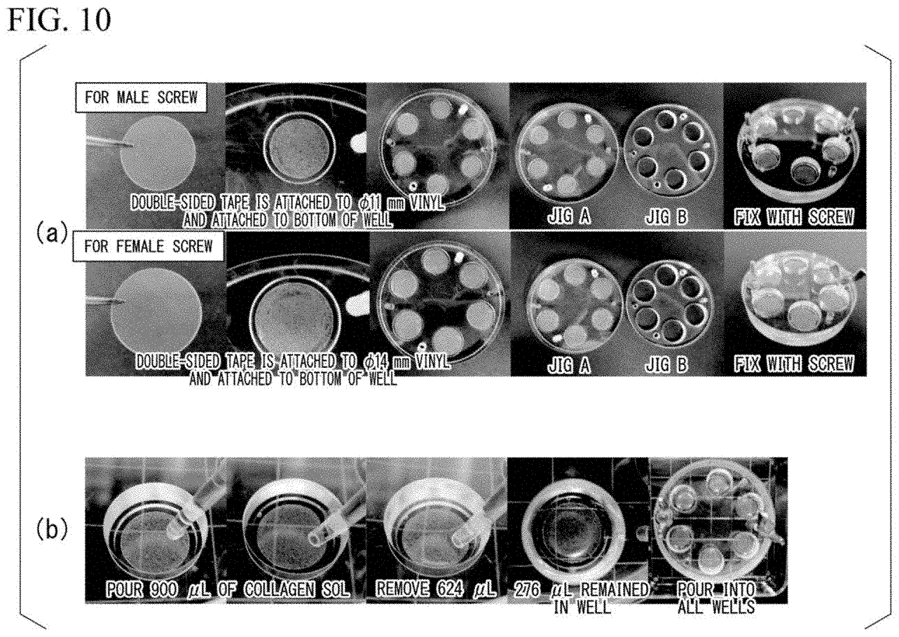

A double-sided tape (double-sided tape that can be peeled off) was attached to φ11 mm and φ14 mm vinyls, and it was attached to wells (for a male screw: φ13.25 mm→1.378 cm², for a female screw: φ16.3 mm→2.086 cm²) of each jig A (base jig) for a male screw and for a female screw and bonded. The φ11 mm vinyl and the φ14 mm vinyl were attached to all 6 wells, and the jig A (base jig) and a jig B (penetration jig) were combined with positioning pins and fixed with screws (refer to FIG. 10(a)). While holding the combined jig firmly, its surroundings were covered with a parafilm.

### (Preparation of Collagen Sol)

3.5 mL of a bovine serum-containing culture medium (10% FBS, 20 mM HEPES, 100 units/mL penicillin, 100 μg/mL streptomycin-containing DMEM) was dispensed into a 50 mL conical tube on ice, and 3.5 mL of a bovine native collagen solution (Koken, I-AC, collagen concentration of 0.5%) was then added thereto, and pipetting was performed 3 times to prepare a uniform 0.25% collagen sol. 900 μL of collagen sol was poured onto the vinyl of the male screw jig and spread over the entire well, 624 μL was then removed and 276 μL remained in the well (refer to FIG. 10(b)). This operation was performed for all 6 wells.

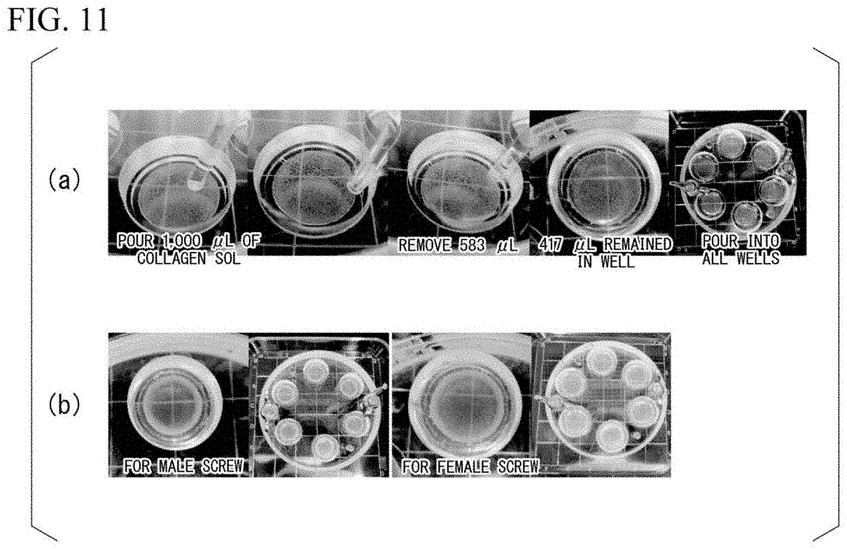

3.5 mL of a bovine serum-containing culture medium (10% FBS, 20 mM HEPES, 100 units/mL penicillin, 100 μg/mL streptomycin-containing DMEM) was dispensed into a 50 mL conical tube on ice, and 3.5 mL of a bovine native collagen solution (Koken, I-AC, collagen concentration of 0.5%) was then added thereto, and pipetting was performed 3 times to prepare a uniform 0.25% collagen sol. 1,000 μL of collagen sol was poured onto the vinyl of the female screw jig and spread over the entire well, 583 μL was then removed and 417 μL remained in the well (refer to FIG. 11(a)). This operation was performed for all 6 wells.

### (Gelation)

The prepared sample was put into a 5% CO₂ incubator set at 37° C. for 2 hours and gelled (refer to FIG. 11(b)).

### (Vitrification)

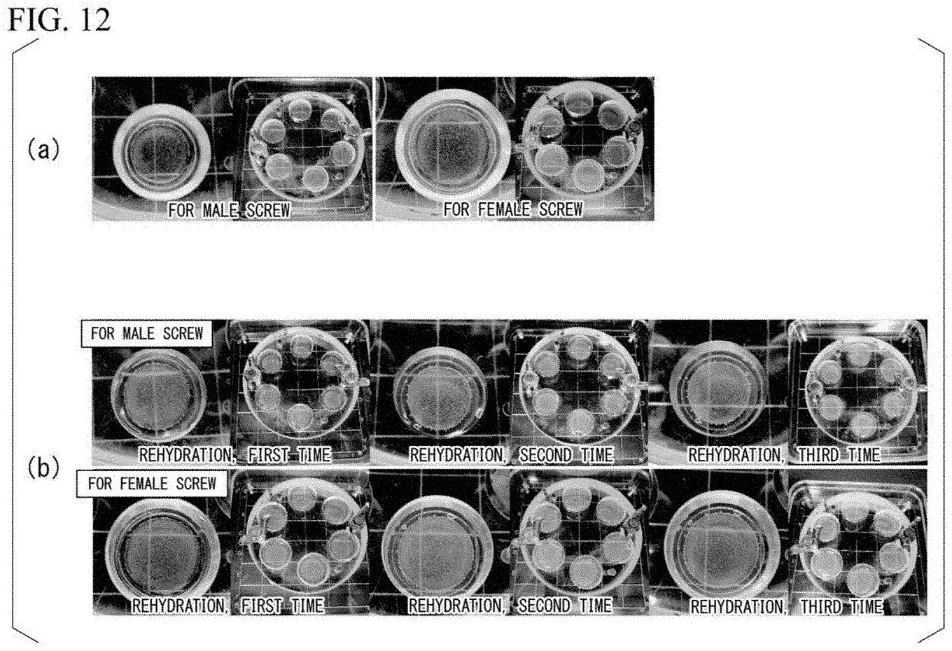

2 hours after gelation, the sample was put into an air dryer in a constant temperature and constant humidity chamber set at 10° C.·40% RH (refer to FIG. 12(a)).

### (Rehydration)

After the vitrification, the sample was taken out from the air dryer, 1 mL of PBS was added and the sample was rehydrated for 10 minutes (first time). After 10 minutes, PBS was removed, and 1 mL of PBS was added again and left for 10 minutes (second time). The same operation was repeated again (third time), PBS was removed and rehydration was completed (refer to FIG. 12(b)).

### (Re-Vitrification)

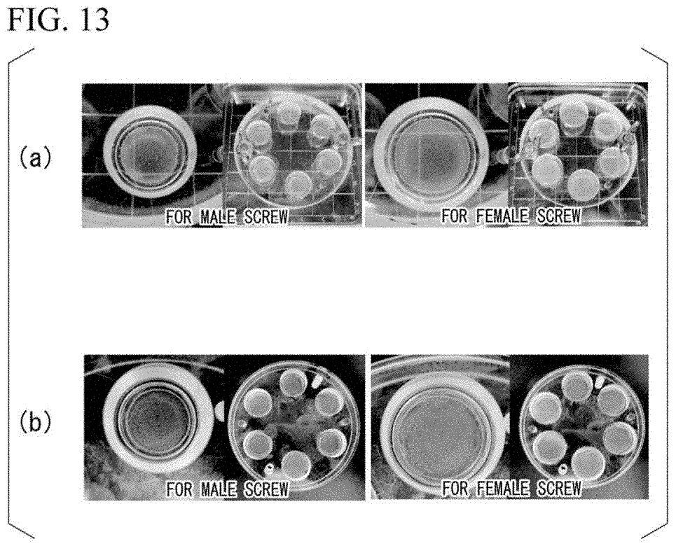

After the rehydration (refer to FIG. 13(a)), the sample was put into an air dryer in a constant temperature and constant humidity chamber set at 10° C.·40% RH, and re-vitrification was performed (refer to FIG. 13(b)).

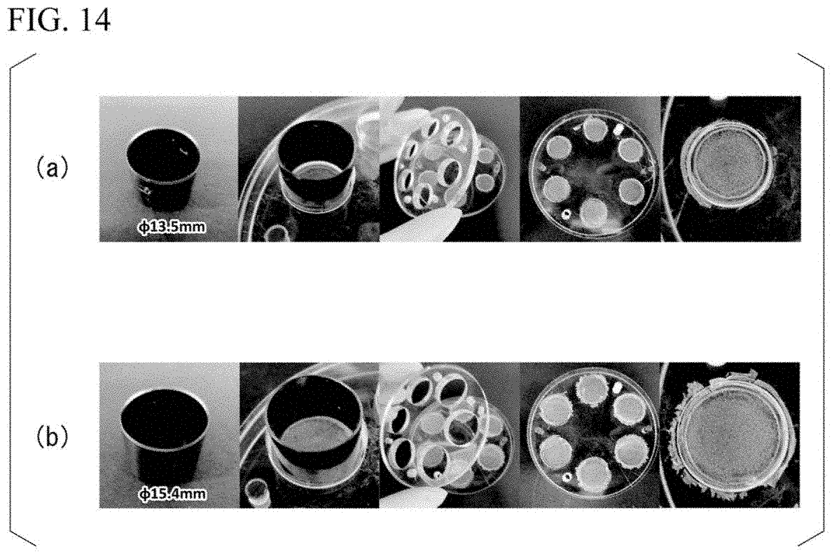

After the re-vitrification, the sample was taken out from the air dryer, a punching blade (φ13.5 mm) was put on the outer upper surface of the well of the jig A (base jig) from the jig B (penetration jig) for a male screw and pressed firmly from above to cut vitrigel dried product formed on the penetrating inner wall surface of the jig B, and cutting was confirmed, and the jig B was then removed from the jig A (refer to FIG. 14(a)). Similarly, a punching blade (φ15.4 mm) was put on the outer upper surface of the well of the jig A (base jig) from the jig B (penetration jig) for a female screw, and cut vitrigel dried product formed on the penetrating inner wall surface of the jig B, and the jig B was removed from the jig A (refer to FIG. 14(b)).

(Production of Screw Type Device with Acrylic Jig)

30 ml of 70% ethanol was put into a 50 ml conical tube, screw type members (a male screw with a tilt angle of 2 degrees and a marking line on an annular tip surface with an inner diameter of φ7.98 mm and an outer diameter of φ11 mm, and a female screw with a tilt angle of 2 degrees and a marking line on an annular tip surface with an inner diameter of φ11.3 mm and an outer diameter of φ14.3 mm) were put in, shaking was performed several times, and then the screw type members were taken out and dried.

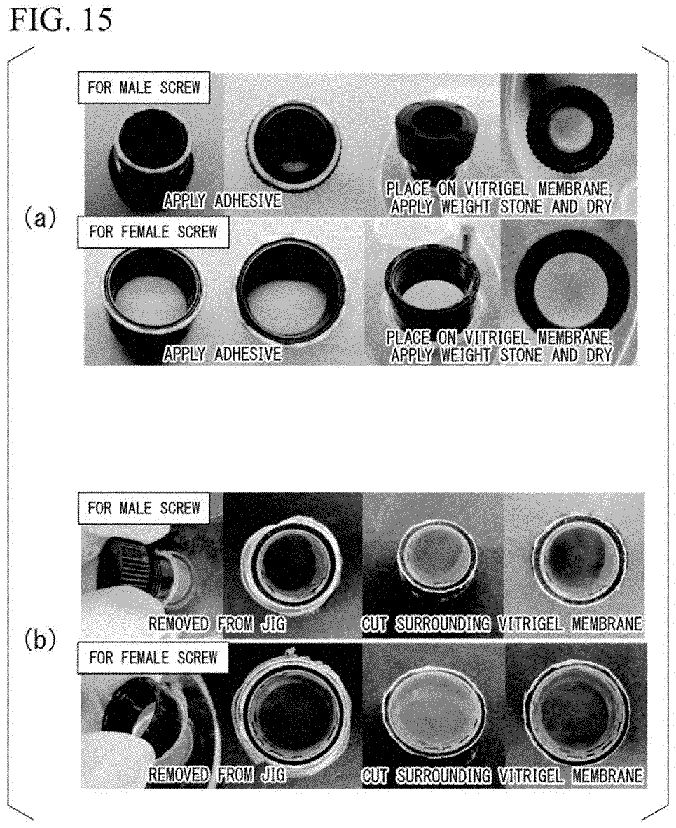

An adhesive was applied to the outside of the marking line on the annular tip surface of each dried screw type member, each screw type member coated with an adhesive was placed on the jig A on which the collagen dried vitrigel membrane product was produced, and attached by placing a weight stone (refer to FIG. 15(a)).

After drying, the collagen dried vitrigel membrane product to which each screw type member was bonded was peeled off from the jig, and the collagen dried vitrigel membrane product protruding to the surroundings was then cut out (refer to FIG. 15(b)).

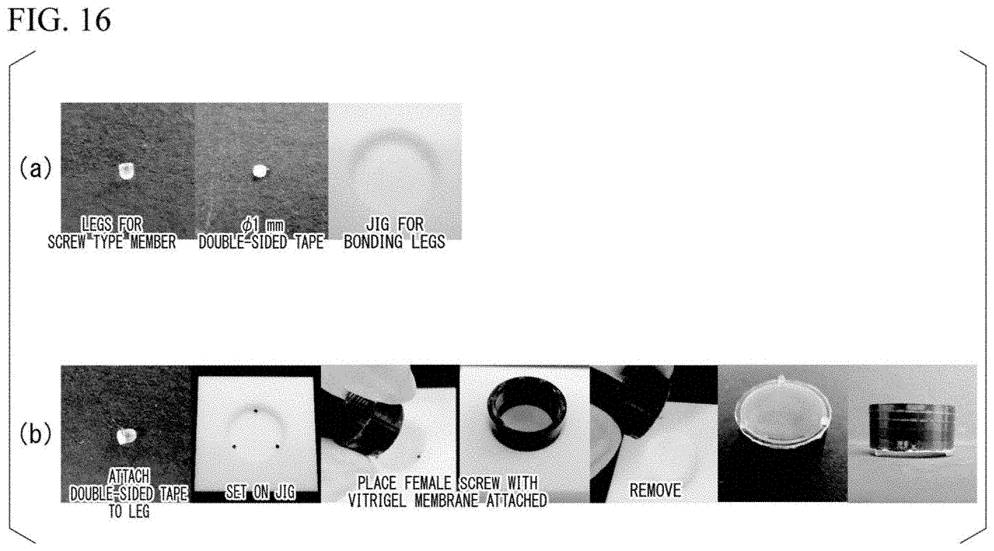

In order to attach legs to the female screw, three legs and a jig for bonding legs were prepared, and a double-sided tape (thin foam base material waterproof double-sided tape: Nitto Denko Corporation) was cut to φ1 mm×3 sheets (refer to FIG. 16(a)).

A φ1 mm double-sided tape was attached to the three legs and placed on a jig for bonding legs, a female screw to which the collagen dried vitrigel membrane product was attached was put thereon and the legs were attached (refer to FIG. 16(b)).

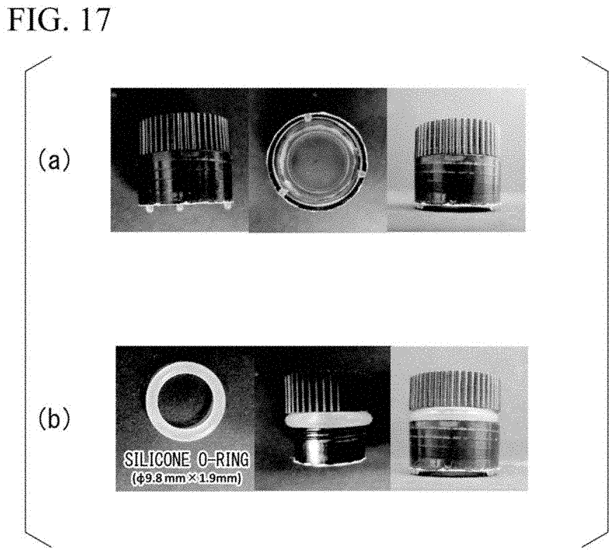

The male screw and the female screw with legs bonded thereto were combined (refer to FIG. 17(a)). In addition, a silicone O-ring was fitted to the male screw (refer to FIG. 17(b)).

Through the above processes, it was possible to obtain a cell culture device in which culture dishes having a collagen dried vitrigel membrane product attached on the bottom surface and having a structure of a male screw and a female screw were able to be mounted with a gap.

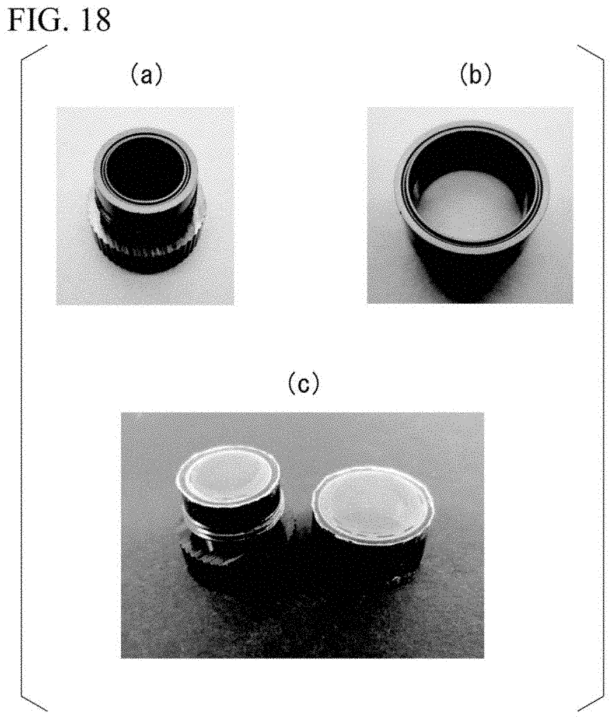

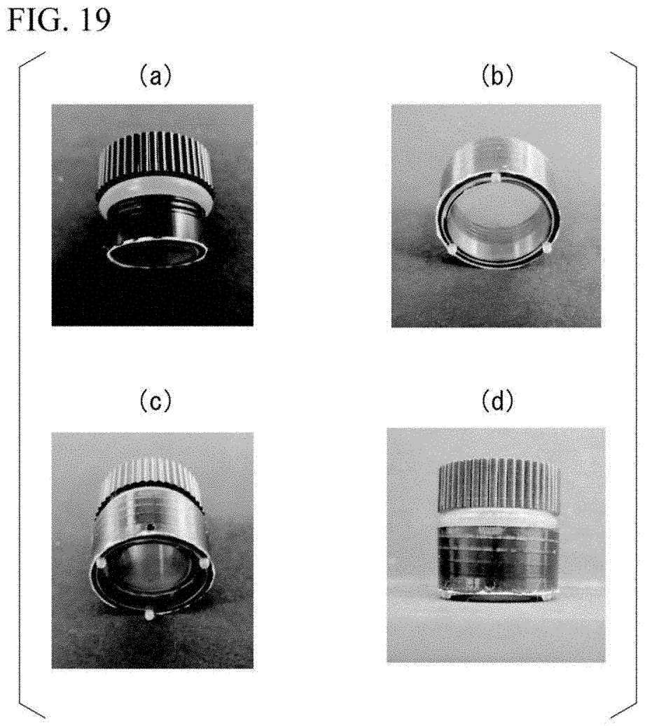

FIG. 18(a) is an image showing the tip surface of the male screw with a marking line and a tilt angle of 2 degrees before the collagen dried vitrigel membrane product was attached. FIG. 18(b) is an image showing the tip surface of the female screw with a marking line and a tilt angle of 2 degrees before the collagen dried vitrigel membrane product was attached. FIG. 18(c) is an image of the male screw and the female screw with the tip surface with a marking line and a tilt angle of 2 degrees to which the collagen dried vitrigel membrane product was attached. FIG. 19(a) is an image of a male screw with a silicone O-ring mounted thereon. FIG. 19(b) is an image of a female screw with legs bonded thereto. FIG. 19(c) is an oblique image of the cell culture device in which a male screw with a silicone O-ring mounted thereon and a female screw with legs bonded thereto were screwed. FIG. 19(d) is a side image of the cell culture device in which a male screw with a silicone O-ring mounted thereon and a female screw with legs bonded thereto were screwed.

### Example 2

### <<Bending Test of Collagen Vitrigel Membrane in Cell Culture Device>>

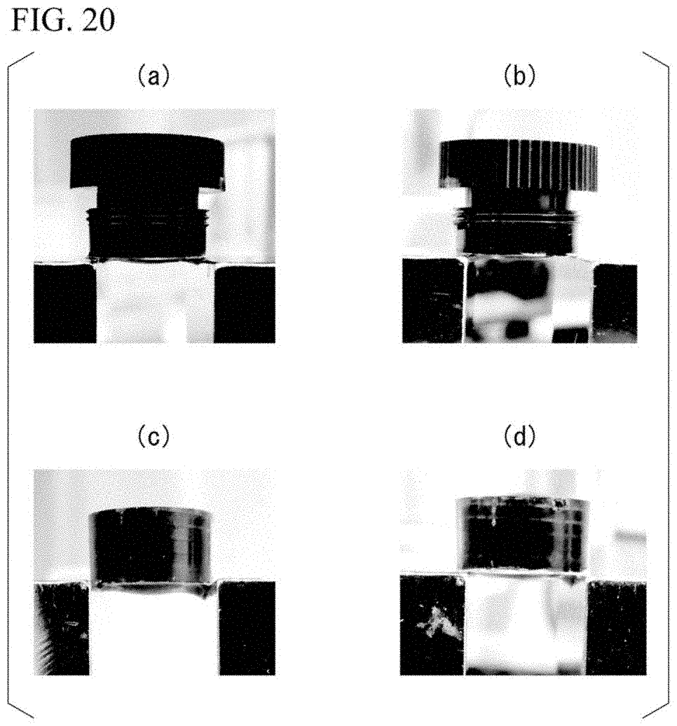

A bending test was performed on a “male screw” and a “female screw” in which a collagen dried vitrigel membrane product was attached to a tip surface with no tilt angle (0 degrees) and a tilt angle of 2 degrees. Specifically, after the collagen dried vitrigel membrane product was rehydrated with PBS, 0.6 ml of PBS was injected into the male screw, 0.8 ml of PBS was injected into the female screw, and the sample was observed from the side. FIG. 20(a) shows a male screw with no tilt angle (0 degrees), FIG. 20(b) shows a male screw with a tilt angle of 2 degrees, FIG. 20(c) shows a female screw with no tilt angle (0 degrees), and FIG. 20(d) shows a female screw with a tilt angle of 2 degrees.

It was confirmed that, in both the “male screw” and the “female screw,” bending of the collagen vitrigel membrane was eliminated on the tip surface with a tilt angle of 2 degrees as compared with the tip surface with no tilt angle (0 degrees).

### INDUSTRIAL APPLICABILITY

According to the present invention, it is possible to provide a cell culture device having excellent handling.

### REFERENCE SIGNS LIST

1 Cell culture device

11 First culture dish

111 First membrane

112 Hole

113 Leg part

12 Second culture dish

121 Second membrane

13 Gap

14 Protection part

15 Lid

16 Third culture dish

2 Hepatocyte culture device

21 First culture dish

22 Second culture dish

211 Second cell culture product

221 First cell culture product

222 Membrane that physiologically active substance is able to permeate

5 Pedestal

3 Oxygen partial pressure control model

31 Lid

32 Oxygen generation mechanism

33 Protection part

4 Human small intestine model

41 Lid

42 Second culture dish

43 First culture dish

1. A cell culture device comprising a first culture dish and a second culture dish,
wherein the first culture dish has a first membrane on the bottom surface,
wherein the second culture dish has a second membrane on the bottom surface, and
wherein the first culture dish and the second culture dish are mounted with a gap of which a height is adjustable therebetween.

2. The cell culture device according to claim 1,
wherein the first culture dish and the second culture dish are mounted by engagement or fitting.

3. The cell culture device according to claim 1,
wherein the bottom surface of the first culture dish has a tapered structure and a marking line for preventing the first membrane from bending, and/or the bottom surface of the second culture dish has a tapered structure and a marking line for preventing the second membrane from bending.

4. The cell culture device according to claim 1,
wherein the first culture dish has a female screw structure,
wherein the second culture dish has a male screw structure, and
wherein the first culture dish and the second culture dish are mounted by screwing.

5. The cell culture device according to claim 1,
wherein the first culture dish has a hole at the height of the gap on a side surface.

6. The cell culture device according to claim 1,
wherein at least one of the first membrane and the second membrane is a liquid-permeable porous membrane or a semipermeable membrane having liquid-tightness in a gas phase and semipermeability in a liquid phase.

7. The cell culture device according to claim 6,
wherein the semipermeable membrane contains a gelling extracellular matrix component.

8. The cell culture device according to claim 7,
wherein the gelling extracellular matrix component is collagen.

9. The cell culture device according to claim 1, further comprising
an Nth culture dish (N is an integer of 3 or more),
wherein the Nth culture dish has an Nth membrane on the bottom surface, and
wherein the (N−1)th culture dish and the Nth culture dish are mounted with a gap of which a height is adjustable therebetween.

10. The cell culture device according to claim 1,
wherein a buffer part is provided on the bottom surface of the first culture dish.

11. The cell culture device according to claim 1,
wherein a lid for closing the second culture dish is provided.

12. A tissue type chip comprising the cell culture device according to claim 1 in which at least the first culture dish contains one type of cells.

13. An organ type chip comprising the cell culture device according to claim 1 including a culture dish containing different types of cells.

14. A cell culture method using the cell culture device according to claim 1.

15. A cell transport method using the cell culture device according to claim 1.

16. A hepatocyte culture device that promotes accumulation and excretion of hepatic metabolites in bile canaliculus-like structures, the device comprising:
a first cell culture product containing a plurality of hepatocytes;
a second culture dish which is able to construct bile canaliculus-like structures between hepatocytes in the first cell culture product, and has a membrane that a physiologically active substance is able to permeate on the bottom surface and in which the first cell culture product is accommodated;
a second cell culture product that is able to increase an excretion activity of hepatic metabolites in the first cell culture product; and
a first culture dish in which the second cell culture product is accommodated,
wherein the first cell culture product is placed on the second cell culture product and co-cultured, and
wherein the first culture dish and the second culture dish are mounted with a gap of which a height is adjustable therebetween.

17. The hepatocyte culture device according to claim 16,
wherein the excretion activity of the hepatic metabolites is an activity of excreting hepatic metabolites accumulated in the bile canaliculus-like structures between hepatocytes and in the hepatocytes in the first cell culture product.

18. The hepatocyte culture device according to claim 16,
wherein the hepatocytes are cultured hepatocytes derived from normal liver tissues, liver cancer tissues or stem cells selected from the group consisting of humans, rats, monkeys, apes, cats, dogs, pigs, cows, sheep, horses, chickens and ducks.

19. The hepatocyte culture device according to claim 16,
wherein the hepatocytes are HepG2-NIAS cells (RCB4679 strain).

20. The hepatocyte culture device according to claim 16,
wherein the second cell culture product is a culture product of cells derived from epithelium, mesenchyme or endothelium selected from the group consisting of humans, rats, monkeys, apes, cats, dogs, pigs, cows, sheep, horses, chickens and ducks.

21. The hepatocyte culture device according to claim 16,
wherein a membrane that is permeable to the physiologically active substance contains a gelling extracellular matrix component.

22. The hepatocyte culture device according to claim 21,
wherein the gelling extracellular matrix component is collagen.

23. A human corneal epithelial model, comprising:
a first cell culture product containing a plurality of human corneal epithelial cells;
a first culture dish which has a membrane that a physiologically active substance in the first cell culture product is able to permeate on the bottom surface and in which the first cell culture product is accommodated; and
a second culture dish having a filtration membrane on the bottom surface,
wherein the second culture dish is placed on the first culture dish, and
wherein the first culture dish and the second culture dish are mounted with a gap of which a height is adjustable therebetween.

24. The human corneal epithelial model according to claim 23,
wherein a buffer part is provided on the bottom surface of the first culture dish.

25. The human corneal epithelial model according to claim 23,
wherein a lid for closing the second culture dish is provided.

26. A human small intestine model, comprising
an anaerobic bacterial culture product;
a second culture dish having a sterile filtration membrane on the bottom surface in which the anaerobic bacterial culture product is accommodated;
a small intestine-derived cell culture product;
a first culture dish which has a semipermeable membrane having liquid-tightness in a gas phase and semipermeability in a liquid phase on the bottom surface and in which the small intestine-derived cell culture product is accommodated; and
an oxygen supply mechanism,
wherein the anaerobic bacterial culture product is placed on the small intestine-derived cell culture product, the small intestine-derived cell culture product is placed on the oxygen supply mechanism, and the first culture dish and the second culture dish are mounted with a gap of which a height is adjustable therebetween.

27. The human small intestine model according to claim 26,
wherein the oxygen supply mechanism includes a mechanism that uses oxygen in air or an oxygen generation mechanism.
